(19) United States
(12) Patent Application Publication
(10) Pub. No.: US 2026/0325704 A1
(43) Pub. Date: Oct. 22, 2026

(54) DUAL-NETWORK TISSUE ADHESIVE FOR BIOMEDICAL APPLICATIONS

(71) Applicant: Board of Regents of the University of Nebraska, Lincoln, NE (US)

(72) Inventors: Bin Duan, Omaha, NE (US); Wen Shi, Papillion, NE (US); Wen Xue, Omaha, NE (US)

(21) Appl. No.: 19/115,847

(22) PCT Filed: Oct. 16, 2023
(86) PCT No.: PCT/US2023/035186

§ 371 Date: Mar. 27, 2025

Related U.S. Application Data
(60) Provisional application No. 63/416,714, filed on Oct. 17, 2022.

(51) Int. Cl. A61L 24/04; A61L 24/00; A61L 24/02; A61L 24/10; C08L 5/08

(52) U.S. Cl. CPC A61L 24/043; A61L 24/0015; A61L 24/0047; A61L 24/02; A61L 24/108; C08L 5/08; A61L 2300/412; A61L 2430/32; A61L 2430/40

(57) ABSTRACT

Compositions and methods for treating and repairing nerve transections are provided.

This application claims priority under 35 U.S.C. § 119(e) to U.S. Provisional Patent Application No. 63/416,714, filed Oct. 17, 2022. The foregoing application is incorporated by reference herein.

### FIELD OF THE INVENTION

This application relates to the field of neurology. More specifically, this invention provides compositions and methods for treating and repairing nerve injuries and transections.

### BACKGROUND OF THE INVENTION

Several publications and patent documents are cited throughout the specification in order to describe the state of the art to which this invention pertains. Each of these citations is incorporated herein by reference as though set forth in full.

The peripheral nerve transection, an intractable clinical problem, is usually caused by traumatic injuries or medical conditions (Menorca, et al. (2013) Hand Clinics 29:317). It can significantly affect and lead to the loss of motor activity and sensation in the respective part of the body (Raza, et al. (2020) Life Sciences, 243:117308; Hall, S. (2005) J. Bone Joint Surg., 87:1309). The current clinical standard treatment for peripheral nerve transection is to coapt injured nerves end-to-end using sutures (Lundborg, G. (2000) J. Hand Surg., 25:391; Lee, et al. (2000) JAAOS 8:243). The proximal and distal nerve ends are connected to shorten the axonal reconnection distance through epineural or perineural suturing in a tension-free manner (Ijkema-Paassen, (2004) Biomaterials 25:1583). However, the presence of the foreign suture material leads to intraneural inflammation, increased fibrosis, and scar tissue formation (Ijkema-Paassen, (2004) Biomaterials 25:1583). This further results in misalignment of axons, delayed remyelination, and failed nerve recovery (Isaacs, et al. (2009) J. Brachial Plexus Peripheral Nerve Injury, 4:e98; Lenoble, et al. (1989) Ann. Chirurgie Main, 8:347; Suri, et al. (2002) Neurology India, 50:23). At present, up to 33% of peripheral nerve injuries in clinics exhibit incomplete nerve recovery and poor functional outcomes, including the loss or partial recovery of motor and sensory function, chronic pain, and end target muscle atrophy and weakness (Wang, et al. (2019) Conn. Tissue Res., 60:3). Besides limited efficacy, the microsurgical suturing procedure is time-consuming and introduces multiple needle passages through the nerve epineurium and perineurium (Sames, et al. (1997) Physiol. Res., 46:303). This secondary damage further induces inflammation and aggravates fibrosis (Ijkema-Paassen, (2004) Biomaterials 25:1583; Amoozgar, et al. (2012) Acta Biomater., 8:1849).

Nerve adhesives are promising alternatives to sutures since they provide a fast and convenient way to connect injured nerve ends. By avoiding secondary tissue trauma, nerve adhesive treatment can potentially decrease inflammation and fibrosis. Among them, fibrin glues are commercially available, biocompatible, possess the longest application history, and have been used in human and many animal studies with the belief that they will minimize the induced trauma (Sameem, et al. (2011) Plastic Reconstr. Surg., 127:2381; Martins, et al. (2005) Surg. Neurol., 64:S10; Egloff, et al. (1983) Ann. Chirurgie Main, 2:101). However, in clinical practice, concerns remain about the lack of adequate adhesion strength for fibrin glue treatment alone, due to its weak physical wrapping around the nerve (Grinsell, et al. (2014) BioMed Res. Int., 2014: 698256). A biomechanical study of rabbit sciatic nerve transection repair reported a 4-time inferior load to failure/gapping strength with fibrin glue relative to suture after treatment (Temple, et al. (2004) J. Reconstr. Microsurg., 20:645). Similar inferior load to failure results have been found in a rat sciatic nerve model immediately and 7 days after surgery (Tse, et al. (2012) Hand Clinics, 28:529). Due to its mechanical limitations, in clinical practice, fibrin glue is predominantly used as an adjunct to sutures or to coapt nerves where suturing is not possible, for example, intervertebral foramen (Grinsell, et al. (2014) BioMed Res. Int., 2014:698256). Therefore, there is a critical need for a next-generation nerve adhesive with enhanced adhesion.

Several new designs have been explored to enhance the strength and mechanics of tissue adhesives. Most of them utilize various reactive groups to achieve covalent bonding with tissue surface. The reactive groups typically include N-hydroxysuccinimide (NHS) esters, cyanoacrylates, aldehydes, and catechol (Yuk, et al. (2019) Nature 575:169; Preul, et al. (2003) Neurosurgery 53:1189; Bu, et al. (2019) Adv. Mater., 31:1901580; Bouten, et al. (2014) Progress Polym. Sci., 39:1375; Korde, et al. (2018) Biomaterials Sci., 6:1691; Wang, et al. (2007) Nat. Mater., 6:385; Artzi, et al. (2009) Adv. Mater., 21:3399; Sedo, et al. (2013) Adv. Mater., 25:653). NHS esters react with primary amines in tissues to form amide bonds spontaneously and fast, but its susceptibility to hydrolysis impedes the adhesion stability (Madler, et al. (2009) J. Mass Spectromet., 44:694). Cyanoacrylates, with an alkoxy carbonyl group and a nitrile group in the monomer, achieve strong covalent crosslinking with the tissue during polymerization. However, their clinical use is limited by toxicity concerns (Taboada, et al. (2020) Nat. Rev. Mater., 5:310). Despite their potential for strong bonding with amines and thiols in tissues, the use of aldehydes often raises risks of cytotoxicity (Xu, et al. (2019) Molecules 24:3005). Among them, catechol-based glues, inspired by the strong underwater adhesion of mussels, have generated tremendous research interests in developing tissue adhesives, including nerve adhesives (Guo, et al. (2020) Nanoscale 12:1307; Ahn, et al. (2015) Nat. Com., 6:1; Zhou, et al. (2016) Biomacromolecules 17:622; Han, et al. (2018) Adv. Funct. Mater., 28:1704195). Catechol (Cat) is the sidechain and adhesive moiety of dihydroxyphenylalanine (DOPA) residue, which is believed to be the key component in adhesive mussel foot proteins (Han, et al. (2018) Adv. Funct. Mater., 28:1704195; Zhou, et al. (2020) ACS Appl. Mater. Interfaces 12:18225; Li, et al. (2015) Chem. Comm., 51:9117; Gan, et al. (2019) Nat. Comm., 10:1; Xie, et al. (2020) Adv. Funct. Mater., 30:1909954). After oxidation, the catechol group is converted to quinone, which can self-polymerize through a quinone-quinone complex and bonds with tissues covalently (through nucleophiles, like amines, thiols, and imidazoles) (Lee, et al. (2019) J. Indust. Eng. Chem., 78:34). However, this process has its limitations. First, quinone-quinone self-polymerization is random and inefficient, and the curing time ranges from dozens of minutes to hours, which is beyond the surgical time frame (Zhang, et al. (2020) Chem. Soc. Rev., 49:433; Paez, et al. (2015) Biomacromolecules 16:3811). Second, a large amount of quinone is accumulated during this process, undergoing reversible redox cycling (O'Brien, P. (1991) Chemico-biological Interactions, 80:1). This redox cycling activates reactive oxygen species (ROS), produces reactive semiquinone, increases inflammation, delays axon growth, and is highly cytotoxic (Bolton, et al. (2017) Chem. Res. Toxicol., 30:13; Monks, et al. (1992) Toxicol. Appl. Pharmacol., 112:2). However, nerve adhesives have high requirements on their bioactivity and supportive environment for axonal regrowth, besides the prerequisite of adhesion strength. Therefore, there is still an unmet need for a nerve adhesive that is bioactive, feasible for surgery, and supports the nerve regeneration process.

### SUMMARY OF THE INVENTION

In accordance with the instant invention, adhesives, particularly nerve adhesives, along with precursors, methods of synthesizing, and methods of using are provided. Adhesive precursor compounds are provided wherein the compound comprises a polymer linked to a phenol via a thiourea. In certain embodiments, the adhesive precursor compound is a hyaluronic acid conjugate. In certain embodiments, the polymer is a glycosaminoglycan such as hyaluronic acid. In certain embodiments, the phenol is a hydroxyphenol such as catechol. In certain embodiments, the polymer is conjugated to the thiourea via a linker. In certain embodiments, the phenol is conjugated to the thiourea via a linker. In certain embodiments, the adhesive precursor compound comprises hyaluronic acid conjugated to a thiourea via a linker and a catechol conjugated to other side of the thiourea via a linker. Composition comprising the adhesive precursor and a carrier are also encompassed by the instant invention.

In certain embodiments, the adhesive of the instant invention comprises the adhesive precursor compound and a decellularized tissue matrix, particularly a decellularized peripheral nerve matrix. The adhesive precursor compound may be crosslinked or conjugated with the decellularized tissue matrix. In certain embodiments, the adhesive comprises a hyaluronic acid conjugate crosslinked with decellularized peripheral nerve matrix, wherein the hyaluronic acid conjugate comprises hyaluronic acid conjugated to a catechol via a linker comprising thiourea. The adhesive of the instant invention may further comprise a therapeutic agent (e.g., a neurotropic agent) and or cells (e.g., neurological cells or Schwann cells).

Methods for producing the adhesive of the instant invention are also provided. In certain embodiments, the method comprises mixing the adhesive precursor compound (e.g., a hyaluronic acid conjugate) and a decellularized tissue matrix, particularly a decellularized peripheral nerve matrix, with an oxidizing agent. In certain embodiments, the oxidizing agent is a chemical oxidizer such as sodium periodate. In certain embodiments, the oxidizing agent is an enzymatic oxidizer such as tyrosinase. Kits for synthesizing the adhesive of the instant invention are also provided. In certain embodiments, the kit comprises a first container comprising the adhesive precursor compound (e.g., a hyaluronic acid conjugate) and a second container comprising a decellularized tissue matrix (e.g., decellularized peripheral nerve matrix) and, optionally, a third container comprising the oxidizing agent.

Methods of using the adhesive are also encompassed by the instant invention. Methods for adhering tissue within a subject are provided, wherein the method comprises administering the adhesive to the desired site for adhesion. Methods for treating a peripheral nerve injury such as a transection are also provided. The method comprises administering an adhesive of the instant invention to the site of injury. For example, the adhesive can be applied or contacted to the two ends of the nerve to be joined.

### BRIEF DESCRIPTION OF THE DRAWINGS

FIG. 1A provides a schematic of the synthesis process of hyaluronic acid (HA)-thiourea (TU)-catechol (Cat) (HA-TU-Cat). FIG. 1B provides a schematic illustrating the formation of two interacting networks in dual-network nerve adhesive (DNNA) composed of HA-TU-Cat and decellularized peripheral nerve matrices (DPN). FIG. 1C provides a schematic of the adhesion mechanism of DNNA via hydrogen bonding of neutrophils from DPN and covalent bonding of catechol from HA-TU-Cat to nerve tissues. DPN provides biomimetic proteins to promote axonal growth. FIG. 1D provides graphs of DNA, collagen, and GAG concentrations of native peripheral nerves (NPN) and DPN (n=4). All quantitative data are expressed as the mean±standard deviation. Asterisks indicate statistically significant comparisons, with ****p<0.0001, by Student's t-test.

FIG. 2A provides images showing gelation of 2% HA-Cat, 2% HA-TU-Cat, and DNNA. FIG. 2B provides graphs of UV-vis spectra of HA-TU-Cat and DNNA before and after oxidation over time. Insets: UV-vis spectra of HA-TU-Cat and DNNA in the wavelength range of 300-400 nm. FIG. 2C provides a graph of the storage (G′) and loss (G′) moduli of HA-Cat, HA-TU-Cat, DPN, and DNNA recorded in a time sweep. FIG. 2D provides a graph of G′ of 1% DPN, 1% HA-TU-Cat, and DNNA (left to right) (n=3). FIG. 2E provides an SEM of DNNA hydrogels. FIGS. 2F and 2G provide graphs of the mass loss rate (%) (FIG. 2F) and swelling ratio (%) (FIG. 2G) of 2% HA-Cat, 2% HA-TU-Cat, and DNNA in PBS at 37 °C (n=3, 4). All quantitative data are expressed as the mean±standard deviation. Asterisks indicate statistically significant comparisons, with **p<0.01 and ***p<0.001, by Student's t-test or one-way ANOVA with Tukey's multiple comparisons tests. FIG. 2H provides a graph of the UV-Vis spectra of HA-TU-Cat and DNNA after oxidation for 3 hours. FIG. 2I provides a graph of the gelation time of 2% HA-TU-Cat oxidized by MT from 250 to 2500 U/mL, DNNA (500 U/mL), and HA-Cat (oxidized by 1:1 molar ratio of catechol to NaIO₄) (n=3).

FIG. 3A provides a graph of force-displacement curves of HA-Cat, fibrin, HA-TU-Cat, and DNNA under lap shear measurement through fresh porcine skins. Lap shears measurement consisted of two sheets of 20×30 mm with 10 mm vertical overlap. FIG. 3B provides a graph of shear adhesion strength of HA-Cat, fibrin, HA-TU-Cat, and DNNA (n=3, 4). FIG. 3C provides a graph of force-displacement curves of HA-Cat, fibrin, HA-TU-Cat, and DNNA under tensile measurement through rabbit sciatic nerves. FIG. 3D provides a graph of tensile adhesion strength of HA-Cat, fibrin, HA-TU-Cat, and DNNA (n=3-5). All quantitative data are expressed as the mean±standard deviation. Asterisks indicate statistically significant comparisons, with *p<0.05 and **p<0.01, by Student's t-test or one-way ANOVA with Tukey's multiple comparisons tests.

FIG. 4A provides images of livability of SCs encapsulated in HA-TU-Cat and DNNA hydrogels after 7 days. FIG. 4B provides a graph of the proliferation of Schwann cells (SCs) encapsulated in HA-TU-Cat and DNNA hydrogels tested by CCK8. FIG. 4C provides images of axonal outgrowth from DRGs on HA-TU-Cat and DNNA hydrogels. FIG. 4D provides a graph of the maximum axonal extension distance from dorsal root ganglions (DRGs) on HA-TU-Cat and DNNA hydrogels. 20-30 axons from 4 DRGs were included in each group. All quantitative data are expressed as the mean±standard deviation. Asterisks indicate statistically significant comparisons, ****p<0.0001, by Student's t-test.

FIGS. 5A-5D show in vivo peripheral nerve anastomosis in a rat model and nerve histological evaluation on day 10 postoperatively. FIG. 5A provides a schematic illustrating the transection and anastomosis of rat sciatic nerves through suture, fibrin glue, and DNNA. FIG. 5B shows Masson's Trichrome staining of sham nerve, suture, fibrin, and DNNA treated nerves 10 days postoperatively. FIG. 5C shows H&E staining of sham nerve, suture, fibrin, and DNNA treated nerves 10 days postoperatively. FIG. 5D shows axon and myelin debris determination at the injury site 10 days postoperatively.

FIG. 6A provides a graph of mechanical sensory functions of rats tested by a von Frey test over time (n=6). FIG. 6B provides a graph of thermal sensory functions of rats tested by a Hargreaves test over time (n=6). #: p<0.001 between DNNA and Fibrin; @: p<0.05 between DNNA and Fibrin. FIG. 6C shows results of footprints captured by a rat-walking apparatus, and a graph of the foot pressure ratio calculated as LH/RH at 10 weeks after surgery (n=3). FIG. 6D provides a graph of perpendicular AEP and PEP ratios (LH/RH) demonstrating body wobble (6-10 steps from 3 rats in each group). FIG. 6E provides a graph of stance/swing durations of each group and their ratios of experimental foot to healthy foot (LH/RH, 6-10 steps from 3 rats in each group). All quantitative data are expressed as the mean±standard deviation. Asterisks indicate statistically significant comparisons, with *p<0.05, **p<0.01, and ***p<0.001, by Student's t-test or one-way ANOVA with Tukey's multiple comparisons tests.

FIG. 7A provides images of harvested nerves in the sham, suture, fibrin, and DNNA groups. FIG. 7B provides Masson's Trichrome staining of nerves in each group. FIG. 7C provides images of myofibroblast determination by IF staining. Cells are αSMA and DAPI stained. FIG. 7D provides images of macrophage determination by IF staining. Cells are CD68 and DAPI stained. FIG. 7E provides images of axon and myelin determination by IF staining. Cells are TUBB3, MBP, and DAPI stained. FIG. 7F provides a graph of the semi-quantification of collagen areas in each group (n=4-6). FIG. 7G provides a graph of the semi-quantification of αSMA positive areas in each group (n=4-6). FIG. 7H provides a graph of the semi-quantification of CD68 positive areas in each group (n=4-6). FIG. 7I provides a graph of the semi-quantification of TUBB3 positive areas in each group (n=4-6). FIG. 7J provides a graph nerve width from different groups at week 10 post-surgery calculated from H&E images (n=3). All quantitative data are expressed as the mean±standard deviation.

Asterisks indicate statistically significant comparisons, with *p<0.05, **p<0.01, and ***p<0.001, by Student's t-test or one-way ANOVA with Tukey's multiple comparisons tests.

FIG. 8A provides images of nerve cross-sections stained with toluidine blue and TEM images at the distal site. FIGS. 8B-8E provide graphs of quantitative data analysis by measuring the average area of single myelinated axons (FIG. 8B), the average area of single neural fibers (FIG. 8C), area-based G-ratio (FIG. 8D), and myelin thickness (FIG. 8E), in different groups according to TEM images. 300 axons from 3 rats in each group were included. All quantitative data are expressed as the mean±standard deviation. Asterisks indicate statistically significant comparisons, with *p<0.05, **p<0.01, and ***p<0.001, by Student's t-test or one-way ANOVA with Tukey's multiple comparisons tests.

FIG. 9A provides images (left: contralateral; right: ipsilateral) and corresponding cross-sectional H&E staining of gastrocnemius muscles of the indicated group. FIG. 9B provides a graph of wet gastrocnemius muscle weight ratio (ipsilateral to contralateral, n=6-8). FIG. 9C provides a graph of the average size of single myofiber of gastrocnemius muscles (n=6-8). FIG. 9D provides a graph of the maximum muscle contraction force of the experimental leg (n=5, 6). All quantitative data are expressed as the mean±standard deviation. Asterisks indicate statistically significant comparisons, with *p<0.05, **p<0.01, and **p<0.001, by Student's t-test or one-way ANOVA with Tukey's multiple comparisons tests.

### DETAILED DESCRIPTION OF THE INVENTION

To address the issues of current nerve glues, a novel catechol-based nerve adhesive, dual-network nerve adhesive (DNNA) was developed. Different from conventional catechol-based adhesives, in the DNNA, dopamine-isothiocy anate containing catechol and thiourea (TU) groups was conjugated to hyaluronic acid (HA), a component of the endoneurial extracellular matrix (ECM) (Asher, et al. (1991) J. Neurosci. Res., 28:410; Tona, et al. (1993) J. Histochem. Cytochem., 41:593), to obtain HA-TU-Cat. HA-TU-Cat was then crosslinked with decellularized peripheral nerve matrices (DPN), a natural material with bioactive peptides and growth factors. HA-TU-Cat can also self-polymerize through quinone-thiourea couplings. Unlike the quinone-quinone complex, the quinone-thiourea coupling is efficient, strong, and reduce quinone back to catechol (Xu, et al. (2020) Sci. Transl. Med., 12:eaba8014; Xu, et al. (2016) Biomater. Sci., 4:1726). All of these allow the DNNA to gelate rapidly, achieve strong adhesion, and decrease quinone accumulation. This strong and bioactive catechol-based adhesive can reconnect injured nerves and enhance nerve recovery.

Herein, the fabrication of DNNA for nerve transection treatment is described. Its gelation behavior and physical and chemical performance are also thoroughly characterized. Robust adhesion strength was determined through lap shear and tensile tests ex vivo. The safety and biocompatibility of the adhesive were verified using in vitro Schwann cell (SC) proliferation and dorsal root ganglion (DRG) neurite outgrowth. The in vivo therapeutic efficacy of DNNA was studied using a rat-based sciatic nerve transection model at 10-day and 10-week post-surgery. Systematic investigations in terms of motor and sensory function recovery, nerve reconnection and remyelination, and muscle structure and function were conducted and compared with the clinical suture treatment and commercial fibrin glues. Importantly, the strong adhesion and bioactivity allowed the adhesive to significantly decrease intraneural inflammation and fibrosis, enhance aligned axon connection and remyelination, aid motor and sensory function recovery, as well as muscle contraction, compared to suture and fibrin treatments.

Overall, an innovative and efficacious adhesive hydrogel is provided for the repair and functional recovery of challenging peripheral nerve transections.

In accordance with the instant invention, an adhesive, particularly a nerve adhesive, is provided. In certain embodiments, the adhesive comprises 1) a hyaluronic acid conjugate comprising a thiourea and catechol and 2) decellularized peripheral nerve matrix. In certain embodiments, the adhesive comprises a hyaluronic acid conjugate comprising a thiourea and catechol crosslinked with decellularized peripheral nerve matrix. In certain embodiments, the adhesive is biocompatible. In certain embodiments, the adhesive is biodegradable.

The hyaluronic acid conjugate of the instant invention comprises hyaluronic acid conjugated to thiourea and catechol. The hyaluronic acid can be any molecular weight. In certain embodiments, the hyaluronic acid has a molecular weight of about 4 kDa to about 4000 kDa, about 10 kDa to about 2000 kDa, about 1 kDa to about 1000 kDa, about 50 kDa to about 1000 kDa, about 100 kDa to about 500 kDa, about 200 kDa to about 400 kDa, about 250 kDa to about 350 kDa, or about 300 kDa. In certain embodiments, the number of repeating disaccharide units (n) of the hyaluronic acid is about 10 to about 10,000, about 25 to about 5000, about 2 to about 2500, about 125 to about 2500, about 250 to about 1250, about 500 to about 1000, about 625 to about 875, or about 750. In certain embodiments, the degree of conjugation onto the hyaluronic acid is about 1% to about 100%, about 5% to about 90%, about 10% to about 30%, or about 20%.

While hyaluronic acid is exemplified herein as part of the adhesive, other polymers may be used in place of the hyaluronic acid. In certain embodiments, the polymer comprises a carboxyl group in the monomer. In certain embodiments, the polymer is a glycosaminoglycan (e.g., hyaluronic acid, heparin, chondroitin sulfate, dermatan sulfate, keratin sulfate). In certain embodiments, the polymer is a hydrogel. In certain embodiments, the polymer is natural. In certain embodiments, the polymer is synthetic. In certain embodiments, the polymer is biocompatible. In certain embodiments, the polymer is biodegradable.

While the hyaluronic acid conjugate is generally described herein as conjugated to a catechol, the catechol may be replaced with other phenols, wherein the phenol comprises one or more hydroxyl groups bonded directly to an aromatic hydrocarbon group (e.g., phenyl). In certain embodiments, the phenol comprises two or more hydroxyl groups on a phenyl. In certain embodiments, the phenol is a dihydroxybenzene or hydroxyphenol such as catechol (1,2-dihydroxybenzene), hydroquinone (1,3-dihydroxybenzene), or resorcinol (1,4-dihydroxybenzene). In certain embodiments, the phenol is a trihydroxybenzene or dihydroxyphenol such as pyrogallol (1,2,3-trihydroxybenzene), hydroxyquinol (1,2,4-trihydroxybenzene), or phloroglucinol (1,3,5-trihydroxybenzene).

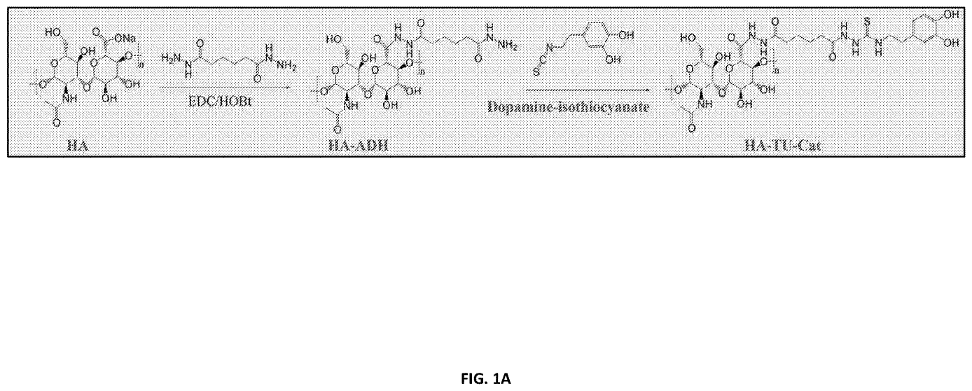

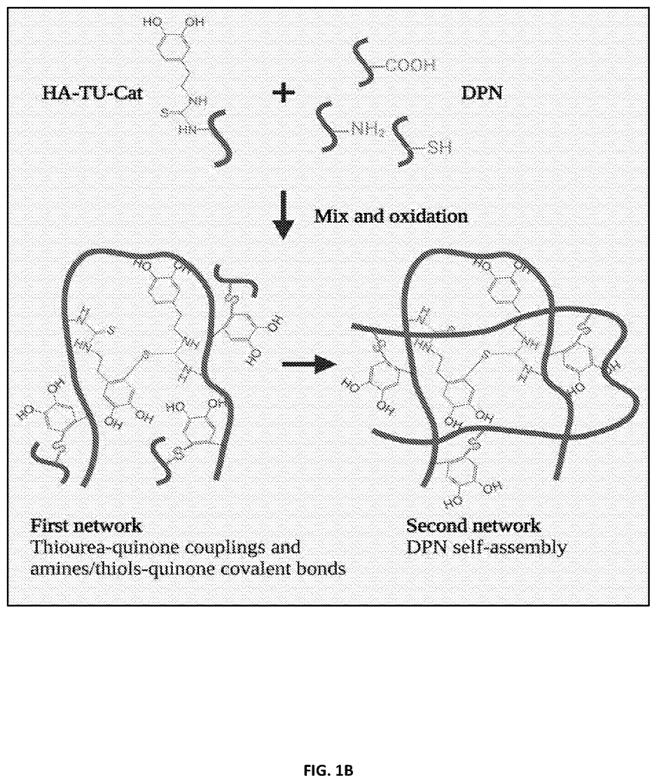

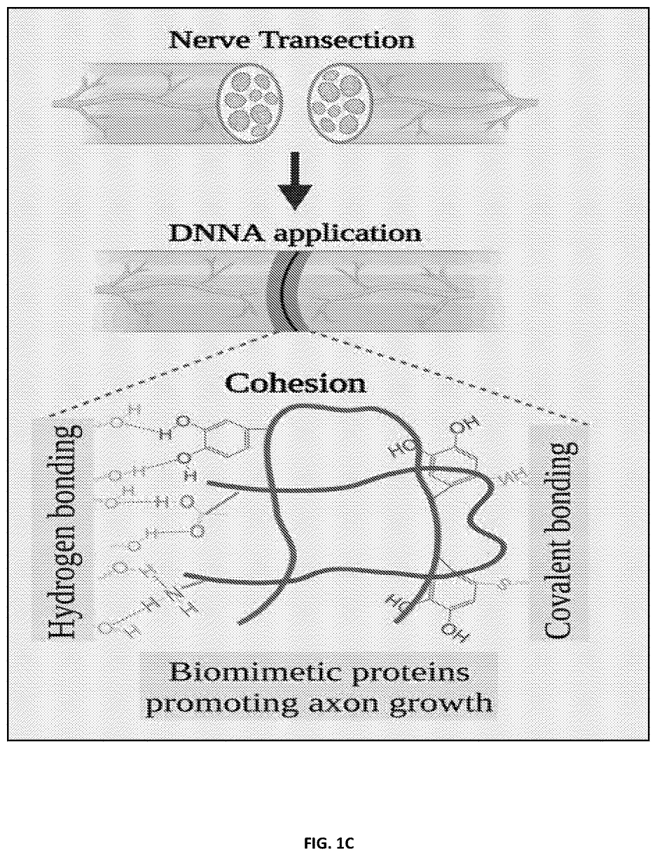

In certain embodiments, the hyaluronic acid conjugate comprises hyaluronic acid conjugated to the catechol via the thiourea group. In certain embodiments, the hyaluronic acid is conjugated to the thiourea via a linker. In certain embodiments, the linker is attached to the hyaluronic acid via a carboxyl group. In certain embodiments, the thiourea is conjugated to the catechol via a linker. In certain embodiments, the thiourea is conjugated to dopamine. In certain embodiments, hyaluronic acid conjugate is a dopamine-isothiocyanate modified hyaluronic acid. In certain embodiments, hyaluronic acid conjugate comprises hyaluronic acid conjugated via a linker to a thiourea conjugated to dopamine. In certain embodiments, hyaluronic acid conjugate is HA-TU-Cat (FIG. 1A).

Generally, a linker is a chemical moiety comprising a covalent bond or a chain of atoms that covalently attaches the two compounds. The linker can be linked to any synthetically feasible position of the compounds, particularly without significantly affecting the activity of the compounds, if applicable. Exemplary linkers may comprise at least one optionally substituted; saturated or unsaturated; linear, branched or cyclic aliphatic group or alkyl group. In a particular embodiment, the linker is an optionally substituted aliphatic or alkyl group. The aliphatic or alkyl group may be unsaturated or saturated and may be substituted with at least one heteroatom (e.g., O, N, or S). In a particular embodiment, the alkyl or aliphatic group comprises about 1 to about 30 carbons, about 1 to about 20 carbons, or about 1 to about 10 carbons (e.g., in the main chain of the alkyl or aliphatic group), which may be substituted with at least one heteroatom (e.g., O, N, or S).

The decellularized peripheral nerve matrix can be derived from any nerve from any animal. In certain embodiments, the animal is a mammal. In certain embodiments, the animal is a pig. In certain embodiments, the animal is a human. In certain embodiments, the nerve is obtained from the subject to be treated. In certain embodiments, the nerve is autologous. In certain embodiments, the nerve is a sciatic nerve. In certain embodiments, the nerve is a porcine sciatic nerve. The decellularized peripheral nerve matrix may have any or all of the following characteristics: (1) the absence of visible nuclei (e.g., as determined by H&E and/or DAPI stained sections); (2) the lack of DNA fragments exceeding 200 bp in length; and (3) <50 ng DNA per mg of matrix. In certain embodiments, the decellularized peripheral nerve matrix comprises collagen and glycosaminoglycan. The decellularized peripheral nerve matrix can be obtained through chemical, physical, and/or enzymatic processes. In certain embodiments, the chemical process comprises ionic detergents (e.g., sodium deoxycholate) and/or nonionic detergents (e.g., Triton™ X 100). In certain embodiments, the enzymatic process involves treatment with DNase, RNase, trypsin, and/or pepsin. In certain embodiments, the decellularized peripheral nerve matrix is sterilized. The decellularized peripheral nerve matrix may be in a solid or a liquid form. While the invention is described using decellularized peripheral nerve matrix, the instant invention also encompasses the use of decellularized tissue matrixes from tissues other than nerves. In certain embodiments, the decellularized tissue matrix is derived from the same type of tissue to be adhered with the adhesive.

The hyaluronic acid conjugate comprising a thiourea and catechol can be crosslinked with the decellularized peripheral nerve matrix to generate the adhesive using an oxidative agent. In certain embodiments, the oxidizing agent is a chemical oxidizing agent (e.g., sodium periodate (NaIO₄). In certain embodiments, the oxidizing agent is an enzymatic oxidizing agent (e.g., tyrosinase such as mushroom tyrosinase).

The adhesive of the instant invention may further comprise or be combined with at least one other functional component. In certain embodiments, the adhesive is combined with or comprises nanoparticles, microparticles, flakes, and/or wires. In certain embodiments, the adhesive is stimulated by electrical, magnetic, ultrasound, and/or light sources.

The adhesive of the instant invention may further comprise at least one therapeutic agent and/or cell. Therapeutic agents of the instant invention may include, without limitation, drugs, pharmaceuticals, biologics, growth factor, cytokines, chemokines, antibodies, antibody fragments, small molecules, peptides, proteins, nucleic acid molecules, DNA, RNA, and other known biologic substances. In certain embodiments, the adhesive further comprises cells such as neurons, Schwann cells, and/or stem cells (e.g., adipose-derived stem cells). In certain embodiments, the therapeutic agent is a neurotropic factor. Examples of neurotropic factors include, without limitation: nerve growth factor and glial derived neurotropic factor. In certain embodiments, the therapeutic agent is an antimicrobial or antibiotic. In certain embodiments, the therapeutic agent is an analgesic.

Antimicrobials may include, without limitation, small molecules, peptides, proteins, DNA, RNA, and other known biologic substances. In a particular embodiment, the antimicrobial is a small molecule. In a particular embodiment, the antimicrobial is an antiviral, antifungal, antibiotic or antibacterial, particularly an antibiotic or antimicrobial peptide (e.g., LL-37 or a fragment or derivative thereof (e.g., those provided in Wang et al. (2019) Adv. Exp. Med. Biol., 1117:215-240, incorporated herein by reference). In a particular embodiment, the antimicrobial is a small molecule antibiotic. Examples of antibiotics include, without limitation, beta-lactams (e.g., penicillin, ampicillin, oxacillin, cloxacillin, methicillin, cephalosporin, etc.), monobactams (e.g., aztreonam, tigemonam, nocardicin A, tabtoxin, etc.), carbapenems (e.g., imipenem, meropenem, ertapenem, doripenem, etc.), cephalosporins (e.g., cefdinir, cefaclor, cephalexin, cefixime, cefepime, etc.), carbacephems, cephamycins, macrolides (e.g., erythromycin, clarithromycin, azithromycin etc.), quinolones or fluoroquinolones (e.g., ciprofloxacin, levofloxacin, ofloxacin, delafloxacin, etc.), tetracyclines (e.g., tetracycline, doxycycline etc.), sulfonamides (e.g., sulfamethoxazole, sulfafuraxole, etc.), aminoglycosides (e.g., gentamicin, neomycin, tobramycin, kanamycin, etc.), oxazolidinones (e.g., linezolid, posizolid, tedizolid, radezolid, contezolid, etc.), lipopeptides (e.g., daptomycin), glycylcyclines (e.g., tigecycline), moenomycins, ansamycins (e.g., rifamycins, rifampin), aminocoumarins (e.g., novobiocin), co-trimoxazoles (e.g., trimethoprim and sulfamethoxazole), lincosamides (e.g., clindamycin and lincomycin), polypeptides (e.g., colistin), and glycopeptides (e.g., vancomycin).

Compositions comprising (1) hyaluronic acid conjugate comprising a thiourea and catechol and/or (2) decellularized peripheral nerve matrix are also encompassed by the instant invention. In certain embodiments, the hyaluronic acid conjugate comprising a thiourea and catechol and/or decellularized peripheral nerve matrix are lyophilized. In certain embodiments, the composition further comprises a carrier (e.g., a pharmaceutically acceptable carrier). In certain embodiments, the composition further comprises at least one therapeutic agent and/or cell, as described above.

In accordance with another aspect of the instant invention, kits are provided. As used herein, the term “kit” refers to a collection of components for use together. For examples, the kits of the instant invention may be for synthesizing the adhesive of the instant invention. The components of the kit may be packaged together. In certain embodiments, the kit comprises 1) hyaluronic acid conjugate comprising a thiourea and catechol and 2) decellularized peripheral nerve matrix. In certain embodiments, the kit further comprises an oxidizing agent. The components may be contained within separate containers. The components may be lyophilized. The components may be in solution.

In certain embodiments, the kit further comprises solutions and/or buffers (e.g., for reconstituting lyophilized components). In certain embodiment, the kits further comprise instruction for using the kit. In certain embodiments, the kit further comprises vials of bottles for mixing of components.

In accordance with another aspect of the instant invention, methods for preparing the adhesive are provided. In certain embodiments, the method comprises contacting or mixing a hyaluronic acid conjugate comprising a thiourea and catechol, a decellularized peripheral nerve matrix, and an oxidizing agent. In certain embodiments, the hyaluronic acid conjugate comprising a thiourea and catechol and the decellularized peripheral nerve matrix are present in a 1:2 to 2:1 ratio. In certain embodiments, the hyaluronic acid conjugate comprising a thiourea and catechol and the decellularized peripheral nerve matrix are present in approximately a 1:1 ratio. In certain embodiments, the method further comprises synthesizing the hyaluronic acid conjugate comprising a thiourea and catechol and/or synthesizing the decellularized peripheral nerve matrix.

In accordance with another aspect of the instant invention, methods of adhering tissue in a subject are provided. Methods of treating peripheral nerve injury (e.g., deformation or transection) are also encompassed by the instant invention. Methods of inhibiting or preventing fluid leakage (e.g., blood, bile, gastrointestinal fluid, or cerebrospinal fluid) are also encompassed by the instant invention. Methods of facilitating wound healing and/or infection prevention are also encompassed by the instant invention. The adhesive of the instant invention can be used with surgical sutures and staples or used in place of with surgical sutures and staples.

The methods comprise administering the adhesive of the instant invention to the desired site. The adhesive of the instant invention can be applied to any tissue including, without limitation: skin, nerves, heart, and intestines. In certain embodiments, the method comprises administering the adhesive to the site of injury such as an injured or transected nerve. The adhesive may be administered to one site or both sites of the tissues to be adhered. In certain embodiments, the method further comprises mixing the hyaluronic acid conjugate comprising a thiourea and catechol and decellularized peripheral nerve matrix with the oxidizing agent prior to administration (e.g., within 30 minutes, 20 minutes, 15 minutes, 10 minutes, or 5 minutes of administration).

The adhesive of the instant invention can be administered to an animal, in particular a mammal, more particularly a human. The compositions of the instant invention may also comprise at least one therapeutic agent (e.g., an antimicrobial) and/or cells. The additional therapeutic agent and/or cells may also be administered in a separate pharmaceutical composition from the compositions of the instant invention.

The pharmaceutical compositions may be administered at the same time or at different times (e.g., sequentially).

The dosage ranges for the administration of the compositions of the invention are those large enough to produce the desired effect. The dosage should not be so large as to cause significant adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The compositions described herein will generally be administered to a patient as a pharmaceutical composition. The term “patient” as used herein refers to human or animal subjects. These compositions may be employed therapeutically, under the guidance of a physician.

The pharmaceutical compositions of the instant invention may be conveniently formulated for administration with any pharmaceutically acceptable carrier(s). For example, the complexes may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents, or suitable mixtures thereof, particularly an aqueous solution. The concentration of the components in the chosen medium may be varied and the medium may be chosen based on the desired route of administration of the pharmaceutical composition. Except insofar as any conventional media or agent is incompatible with the component to be administered, its use in the pharmaceutical composition is contemplated.

Selection of a suitable pharmaceutical composition will also depend upon the mode of administration chosen. For example, the adhesive of the invention may be administered by direct injection. In this instance, a pharmaceutical composition comprises the component dispersed in a medium that is compatible with the site of injection. Pharmaceutical compositions for injection are known in the art.

### Definitions

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., TrisHCl, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

The term “antimicrobials” as used herein indicates a substance that kills or inhibits the growth of microorganisms such as bacteria, fungi, viruses, or protozoans.

As used herein, the term “antibiotic” refers to antibacterial agents for use in mammalian, particularly human, therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

As used herein, the term “subject” refers to an animal, particularly a mammal, particularly a human.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

A “therapeutically effective amount” of a compound or a pharmaceutical composition refers to an amount effective to prevent, inhibit, treat, or lessen the symptoms of a particular disorder or disease. For example, the treatment of an infection herein may refer to reducing, curing, and/or relieving the infection, the symptom(s) of it, or the predisposition towards it.

As used herein, the term “analgesic” refers to an agent that lessens, alleviates, reduces, relieves, or extinguishes pain in an area of a subject's body (i.e., an analgesic has the ability to reduce or eliminate pain and/or the perception of pain).

As used herein, the term “small molecule” refers to a substance or compound that has a relatively low molecular weight (e.g., less than 2,000). Typically, small molecules are organic, but are not proteins, polypeptides, or nucleic acids.

The following example illustrates certain embodiments of the invention. It is not intended to limit the invention in any way.

### EXAMPLE

### Materials and Methods

Study design

This study is dedicated to developing a nerve adhesive with robust adhesion strength and a bioactive composition for effective nerve transection treatment. The DNNA incorporated with catechol groups and decellularized nerve matrices was prepared to promote nerve regeneration and functional recovery of transected sciatic nerves. It was thoroughly characterized in terms of gelation behavior and mechanism, rheology, morphology, and adhesion performance. The in vitro biocompatibility was further tested to illustrate improved axonal outgrowth rendered by the addition of DPN. A sciatic nerve anastomosis model in rats was chosen to rigorously examine and compare the in vivo efficacy of the DNNA with a standard suture treatment and commercial fibrin glue. Nerve regeneration and functional recovery was characterized by sensory and motor behavior analysis every 2 weeks until the end of week 10. The gastrocnemius muscle contractile force and structure were determined to directly evaluate muscle atrophy. The histology, including fibrosis, foreign body reactions, axon reconnection, and myelination of injured nerves from the early stage (day 10) and late stage (week 10) was also examined.

### Synthesis and Characterization of HA-Cat

HA-Cat was synthesized according to protocol (Zhou, et al. (2020) ACS Appl.

Mater. Interfaces 12:18225). Briefly, 0.5 g of HA powder (300 kDa, Bloomage Biotechnology Corporation Limited) and 0.25 g of NaIO₄ (Alfa Aesar) were dissolved in 50 mL of phosphate buffered saline (PBS, pH=5) and stirred for 5 hours at room temperature (avoid light). Afterwards, 0.5 mL of ethylene glycol (Sigma-Aldrich) were added and stirred for another 1 hour to neutralize the NaIO4. The mixed solution was then dialyzed against deionized (DI) water for 2 days and lyophilized to obtain the aldehyde-modified HA.

To get dopamine conjugated HA, 0.5 g of aldehyde-modified HA were dissolved in PBS (pH=5). 0.5 g of dopamine hydrochloride (Combi-Blocks) were then added and stirred for 10 hours at room temperature. After dialysis against DI water and lyophilization, the ¹H NMR spectrum was recorded on a 500 MHz Bruker NMR system. The DS was determined by the ratio of the integral of catechol methylene protons from the conjugated dopamine (between 2.8 ppm, —CH₂ ) to the integral of the HA methyl proton peak (at 2.0 ppm, —CH₃ ) using the equation DS=3×A_{2.8}/(2×A_{2.0}).

### Synthesis and Characterization of HA-TU-Cat

HA-ADH was first synthesized. Briefly, 0.5 g of HA was dissolved in 100 ml of DI water. 1.2 g of 1-ethyl-3-(3-dimethylaminopropyl) carbodiimide hydrochloride (EDC, Combi-blocks), 1 g of HOBt hydrate (Oakwood Chemical), and 17 g of adipic dihydrazide (Tokyo Chemical Industry) were added. The pH of mixed solution was then adjusted to 6 and stirred at room temperature for 24 hours. After the reactions were finished, the solution was dialyzed against sodium chloride (NaCl, Fisher Chemical) for 3 days and DI water for another 3 days and then lyophilized. The ¹H NMR was examined to confirm its successful synthesis.

Dopamine-isothiocyanate was then synthesized (Xu, et al. (2016) Biomaterials Sci., 4:1726; Medintz, et al. (2010) Nat. Mater., 9:676; Ji, et al. (2012) JACS 134:6006). Briefly, 6 g of dopamine hydrochloride were dissolved in 80 mL of tetrahydrofuran (Fisher Chemical) with 5.8 mL of triethylamine (Sigma-Aldrich). 82 mL of methanol (Fisher Chemical) were slowly added to help dissolution under the ice-cold conditions and nitrogen atmosphere. After forming a clear, colorless solution, 10 mL of carbon disulfide (Sigma-Aldrich) was added dropwise. The solution was left stirring for 2 hours at 5-10 °C for reaction. The mixture was then warmed to room temperature while stirring for a further 10 to 12 hours. The reaction mixture was cooled again to 5 °C, and 10 mL of 30 % hydrogen peroxide (H₂O₂, Fisher Chemical) were slowly added and stirred for 10 min under nitrogen atmosphere. The mixture was then neutralized with 37% hydrochloric acid (HCl, Fisher Chemical, 2 mL). The resulting mixture was filtered and concentrated under vacuum. Then 50 mL of DI water and 50 mL of ethyl acetate (Alfa Aesar) were added into the mixture, and the crude product was purified by a silica column chromatograph (300-400 mesh, Acros Organics) using a chloroform/ethyl acetate (3:1, v/v) mixture. The final product was obtained as a white powder, and the ¹H NMR was recorded.

HA-TU-Cat was then obtained by dissolving 0.5 g of HA-ADH in 50 mL of DI water and reacting it with 0.3 g of dopamine-isothiocyanate powder in 50 mL of dimethyl sulfoxide (DMSO) for 3 days at room temperature (FIG. 1A). The mixture was purified by dialysis against NaCl solution for 3 days and DI water for another 3 days. ¹H NMR was conducted, and the degree of substitution was calculated by the ratio of the integral of aromatic protons from the conjugated catechol group (between 6.5~7.0 ppm, —C₆H₃) to the integral of the HA methyl proton peak (at 2.0 ppm, —CH₃).

### Decellularization of Porcine Peripheral Nerves

Fresh porcine sciatic nerves (freshly isolated from five domestic pigs) were obtained from Tissue Source, LLC. Before decellularization, the fat tissues around the nerves were cleaned and removed. Then, the nerves were subjected to a combined chemical and enzymatic decellularization process (Philips, et al. (2018) Ann. Biomed. Engr., 46:1921). Briefly, fresh nerves were stirred in 50 mM Tris buffer (pH=8) overnight and then in 1% Triton™-X 100 in 50 mM Tris buffer solution at 4 °C for 24 hours. The enzymatic treatment with 40 units/mL DNase (04536282001, Roche), 20 mg/L RNase (R4875, Sigma-Aldrich), and 0.01% Trypsin (Sigma-Aldrich) in Hank's Balanced Salt Solution (HBSS) was performed for 4 hours at 37 °C. Then the nerves were stirred overnight in 1% Triton™-X 100 in 50 mM Tris buffer at 4 °C. After lyophilization, the decellularized nerves were sterilized in 70% ethanol for 2 hours and rinsed thoroughly with PBS. They were then cut into small pieces and digested in 1 mg/mL pepsin (Sigma-Aldrich) in 0.01 N HCl solution (with 20 mg/mL decellularized nerve). After constant agitation for 72 hours at room temperature, the pH of the solution was adjusted to 7.5 by adding 1 N sodium hydroxide (NaOH, Fisher Chemical), and the salt concentration was adjusted by adding 10×PBS ( 1/10 of the final volume). This pre-gel solution was stored at −20 °C until use.

Histological sectioning and H&E staining were performed to verify the decellularization results. Native nerves and decellularized nerves were fixed in 10% formalin buffer for 4 hours. After washing with PBS thrice, they were embedded in paraffin, sectioned, and stained by H&E. To determine the DNA, collagen, and GAG contents, native and decellularized nerve tissues were digested in 0.3 mg/mL papain solution (Sigma-Aldrich). The residual amount of DNA of NPNs and DPNs were quantitatively evaluated by a Quant-iT™ picoGreen™ kit (Invitrogen) following the manufacturer's procedures. The collagen contents were measured through a hydroxyproline assay, and the GAG contents were measured using a 1,9-dimethylmethylene blue (DMMB) assay (Xue, et al. (2022) ACS Applied Mater. Interfaces 14:8693; Duan, et al. (2014) Acta Biomaterialia 10:1836).

### Preparation of HA-Cat and DNNA Hydrogels

For HA-Cat adhesive hydrogel preparation, 2% HA-Cat was dissolved in PBS and mixed with MT at 37 °C (2500 U/mL, Sigma-Aldrich) or NaIO₄ at room temperature for 30 minutes (1:1 molar ratio of dopamine to NaIO 4). Note that the MT is a gentle and biocompatible oxidizing enzyme, compared to chemical oxidants like NaIO₄. However, to ensure successful gelation of the HA-Cat, NaIO₄ was also utilized here. 2% HA-TU-Cat was also dissolved in PBS and mixed with MT (500 U/mL). The solution was then incubated at 37 °C for 5 minutes to gelate.

To prepare the DNNA, 2% HA-TU-Cat in PBS was mixed with an equal volume of 20 mg/mL DPN pre-gel solution at 4 °C. MT (500 U/mL) was then added into the mixed solution and vortexed immediately. The whole solution was kept at 37 °C for 5 minutes to form physically and chemically double interacted hydrogel networks.

The influence of the DPN addition into HA-TU-Cat on its crosslinking mechanism was examined by UV-vis spectroscopy. The spectrum of the HA-TU-Cat and DNNA before and after oxidation was recorded over time in the wavelength range from 260 nm to 600 nm.

### Rheological Performance of Nerve Adhesives

The rheological performances of 1% DPN, 2% HA-Cat, 1% and 2% HA-TU-Cat, and DNNA were tested on a rheometer (HR-2, TA Instruments). The plate diameter was 20 mm, and the gap was 0.6 mm. The initial temperature of the plate was set as 4 °C to ensure liquidity of the pre-gel solutions. Then the plate temperature was rapidly increased to 37 °C at the rate of 100 °C /minute, followed by a dynamic time sweep at the frequency of 1 Hz and 1% strain. The G′ and G″ of the hydrogels were recorded.

### Morphology of Nerve Adhesives

For the morphological observation, 2% HA-Cat, 2% HA-TU-Cat, and DNNA were lyophilized and quenched in liquid nitrogen. Gold sputter coating was conducted, and they were observed by SEM (FEI Quanta 200) at 25 kV.

### In Vitro Stability and Swelling of Nerve Adhesives

For the stability evaluation, 50 μL of 2% HA-Cat, 2% HA-TU-Cat, and DNNA solutions were added into silicone molds with 6 mm diameters and formed hydrogels at 37 °C for 30 minutes. They were then incubated in PBS (pH=7.4) at 37 °C. At predetermined time points, the hydrogels were taken out of the PBS, rinsed with DI water, and lyophilized. The mass loss rate (%) was determined as: (original lyophilized hydrogel mass-lyophilized hydrogel mass at each time point)/ original lyophilized hydrogel mass×100%.

As for the hydrogel swelling, lyophilized hydrogel samples were prepared and immersed in PBS (pH=7.4) at 37 °C. At specific time points, the mass of rehydrated hydrogels was measured. The swelling ratio (%) was calculated as: (rehydrated hydrogel mass at each time point-original lyophilized hydrogel mass)/ original lyophilized hydrogel mass×100%.

### Adhesion Performances of Nerve Adhesives

The adhesion performances of HA-Cat, HA-TU-Cat, DNNA, and commercial fibrin glues (Tisseel) were evaluated by lap shear tests through porcine skins and tensile tests through rabbit nerves. The lap shear tests of nerve adhesives were conducted according to the ASTM F2255-05 standard (Testing, Materials, Standard Test Method for Strength Properties of Tissue Adhesives in Lap-Shear by Tension Loading, ASTM International, 2005). Fresh porcine skins were bought from Nebraska Scientific and rinsed in PBS before removal of their fat layers. They were then cut into pieces of 20 mm in width and 40 mm in length. Then 0.2 mL of 2% HA-Cat/NaIO₄ (1:1 molar ratio), 2% HA-TU-Cat/MT (500 U/mL), 2% DNNA/MT (500 U/mL) solutions, or fibrin glue were applied onto the inner surface of one piece of porcine skin. It was covered by another porcine skin immediately, and the bonding area was 20 mm×10 mm. All samples were kept at 37 °C for 30 minutes before measurements. Tests were performed on the Cellscale Univert tensile machine at a 6 mm/minute take-up rate until failure. Force-displacement curves were recorded, and the adhesion strength was calculated as the failure force divided by the bonding area.

A tensile adhesion test determines the force required to pull adhesives off nerve surface. Rabbit sciatic nerves were collected from New Zealand White rabbits after euthanasia. The rabbits were euthanized subjects from another research protocol, which was approved by the Institutional Animal Care and Use Committee (IACUC) of the University of Nebraska Medical Center (UNMC). Rabbit nerves with lengths around 50-60 mm were cut into two halves. 20 μL of nerve adhesives prepared as formally mentioned were applied to one end of the cut nerve and connected with the other end immediately. All samples were kept at 37 °C for 30 minutes before tensile evaluations. Tests were conducted on the Cellscale Univert tensile machine at a 6 mm/minute take-up rate until failure.

To further qualitatively demonstrate the utilization and feasibility of the DNNA, the isolated rabbit sciatic nerve was transected into three parts and reconnected with the DNNA at 37 °C for 5 minutes. In situ application on rabbits (after euthanasia) was conducted by connecting transected sciatic nerves for 5 minutes at room temperature. To evaluate adhesive performance of the DNNA under wet conditions, DNNA mixed with green food dye was first gelated on porcine skins and then twisted and immersed in PBS for 30 minutes. In addition, the DNNA was gelated on porcine skins and glass slides and was continuously flushed under water for 5 minutes.

### In Vitro SC and DRG Culture in Nerve Adhesives

Rat SCs were isolated from fresh rat sciatic nerves, as previously reported. [32]4×10⁵ SCs/mL were suspended in 2% HA-TU-Cat/MT (500 U/mL) and DNNA/MT (500 U/mL) solution. Because NaIO₄ is cytotoxic, the 2% HA-Cat/NaIO₄ group was not included in the in vitro cell evaluations or in vivo experiments. 50 μL of SCs/adhesives mixed solutions were then added into silicone molds with 6 mm diameters and formed hydrogels at 37 °C for 30 minutes. Afterwards, SC culture medium, consisting of DMEM/low glucose medium (Cytiva HyClone™), 10% fetal bovine serum (FBS, Gibco), and 1% Pen/Strep (P/S, Gibco), was added and cultured for 7 days. A Live/Dead assay (Invitrogen) was applied to evaluate cell viability after 7 days of culture. The samples were imaged by a confocal laser scanning microscope (CLSM, LSM 710, Zeiss). The SC proliferation was determined at days 3 and 7 by a Cell Counting Kit 8 (CCK8, Abcam), following the manufacturer's protocol.

DRGs were dissected from 15-day-old mice (C 57BL/6 background) from another research protocol, which was approved by the IACUC of UNMC. The dissected DRGs (from L4-L6 lumbar segments) were placed on 2% HA-TU-Cat/MT (500 U/mL) and 2% DNNA/MT (500 U/mL) hydrogels on glass slides (incubated at 37 °C for 30 minutes). After allowing DRG attachment on the adhesives at 37 °C for 1 hour, DRG growth medium, consisting of DMEM/F12 medium (Cytiva HyClone™), 10% FBS, 1% P/S, and 10 ng/mL nerve growth factor (NGF, Peprotech), was added and cultured for 7 days. For IF staining of neurites from DRGs, the constructs were fixed in 4% paraformaldehyde (PFA) at 4 °C for 4 hours and then rinsed with PBS. The samples were blocked by 1% bovine serum albumin (BSA) overnight at 4 °C. They were then incubated with primary antibodies to TUBB3 (1:400, rabbit, BioLegend) with 0.04% Triton™ X-100 in 1% BSA overnight at 4 °C. Alexa Fluor™ 568 goat anti-rabbit antibody (1:100 in 1% BSA) was then added and cultured for 2 hours at room temperature. Finally, the cell nuclei were stained by DAPI (1:1000 in PBS) for 10 minutes. Imaging was performed with a 710 CLSM. The lengths of DRG neurites were quantified by the NeuronJ Plugin of Image J software (Xue, et al. (2022) ACS Appl. Mater. Interfaces 14:8693).

### In Vivo Peripheral Nerve Anastomosis

Animal experiments were approved by the IACUU of UNMC. A total of 40 female Sprague-Dawley rats (aged 9 weeks with weights ranging 170-250 g) were randomly divided into four groups: sham group (nerve was exposed without transection); suture group (nerve was transected and anastomosed with sutures); fibrin glue group (nerve was transected and anastomosed using commercial fibrin glue from Tisseel); DNNA group (nerve was transected and anastomosed with DNNA). Rats were anesthetized with isoflurane gas throughout the procedure. A skin incision was made parallel to the femur on the right side to expose the sciatic nerve. Afterwards, the sciatic nerve was transected with microsurgery scissors carefully under an operating microscope. For the suture group, end-to-end coaption of cut nerves was performed by two-stiches suturing (10-0 polyglycolic acid, Shapoint). For the fibrin glue and DNNA groups, 50 μL of the adhesives were applied onto the anastomosis site and cured for 5 minutes. Afterwards, the muscles and skins were sutured separately, and the rats received routine postoperative care and were kept in standard conditions. Random selected rats from suture, fibrin, and the DNNA groups were euthanized on day 10 for nerve collection. At the end of the experiment (week 10), rats from each group were anesthetized for muscle contraction testing. After animal euthanasia, the ipsilateral gastrocnemius muscles, sciatic nerves, L4/L5 DRGs, and spinal cords were harvested for further physiological and histological analysis.

### Evaluation of Sensory and Motor Function Recoveries

The sensory and motor functions of the hindlimbs were evaluated based on behavior analyses, including von Frey, Hargreaves, and gait analysis tests, every two weeks post-surgery (weeks 2, 4, 6, 8, 10). To test the mechanical sensitivity, the rats were confined in boxes with a metal-mesh floor. After 30 minutes of habituation, their experimental hind paws were stimulated with von Frey filaments (Aesthesio) in incremental pressures (maximum of 50 g) (Wang, et al. (2022) Biomaterials 280:121251; Kong, et al. (2021) Appl. Mater. Today 24:101090). Immediate foot lifting or licking was regarded as a withdrawal response. Five trials were conducted for each rat at intervals of 5 minutes, and the mechanical withdrawal threshold (g) was calculated as the mean of five consecutive trials. Thermal sensitivity was tested using a Hargreaves radiant heat apparatus (Ugo Basile Instrument), with an intensity of 45% and a cut-off time set as 30 seconds to avoid tissue damage. The latency of licking, withdraw, or shaking was recorded, and six trials for each rat were conducted with an interval of 10 minutes. To evaluate the rats' locomotor functions, a homemade rat-walking apparatus based on a prototype of a MouseWalker system was utilized to analyze gait behaviors (Mendes, et al. (2015) BMC Biol., 13:1). The apparatus was composed of a red backlit top panel, acrylic glass surrounded by green lights, a walking corridor above the glass, and a mirror at a 45-degree angle below the glass. Each rat received the training to walk from the entrance to the exit of the walking corridor before the test until there were no distractions or hesitation behaviors observed during the walking. During the test, rat walking was observed through the mirror and recorded by using a GoPro camera. The videos were uploaded into and run in the MouseWalker program in Matlab (Mathworks). The gait parameters for experimental hindlimbs of foot pressure; gait tracking information, including AEP and PEP; and stance and swing durations were quantified and compared to these of contralateral healthy hindlimbs.

### Muscle Characterization and Contractile Force

The rats were anesthetized with urethane (800 mg/kg ip) and a-chloralose (40 mg/kg ip) at week 10 to characterize the contractile force of gastrocnemius muscle. The gastrocnemius muscle was isolated, and its distal end was connected to a wide range force transducer (1030/D; AD Instruments, Colorado Springs). A bipolar hooked stimulating electrode was applied to the exposed sciatic nerve. To induce the tetanic contractile force of the gastrocnemius muscle, a constant-current stimulation was generated by a Grass S9 stimulator (Grass Instruments, West Warwick, RI) at three times the motor threshold (defined as the minimum current required to produce a muscle twitch) with a pulse duration of 0.1 ms at 50 Hz. After the measurement of muscle contraction force, the rats were sacrificed to harvest gastrocnemius muscles and measure their wet weights. Then the gastrocnemius muscles were fixed in 4% PFA overnight at 4 °C, embedded into paraffin, and sectioned (4 μm). H&E staining was conducted to evaluate the atrophy of myofibers in the gastrocnemius muscles by calculating their cross-sectional area by using ImageJ software. Images were taken from 3 randomly selected fields of each rat, and 3 rats in each group were analyzed.

### Histological and Morphological Analyses

At day 10 post-surgery, sciatic nerves in the suture, fibrin, and DNNA groups were harvested and fixed in 4% PFA overnight at 4 °C. They were then embedded into paraffin, sectioned (4 μm), and subjected to H&E and Masson's Trichrome staining according to the standard protocols in the Tissue Science Facility at UNMC. For the IF staining, slides were deparaffinized and rehydrated, followed by antigen retrieval in heated citrate buffer for 20 minutes (citrate buffer solution, pH 6.0 at 100 °C) (Janjic, et al. (2018) J. Neuroimmunol., 318:72). Nonspecific antibody binding was blocked by 5% goat serum solution for 2 hours at room temperature, followed by overnight incubation at 4 °C with primary antibody in 0.04% Triton™ X-100 and 5% goat serum. Then corresponding secondary antibodies were added and incubated for 2 hours at room temperature. Samples were imaged by a 710 CLSM after nucleus staining with DAPI for 10 minutes. The primary antibodies used included TUBB3, MBP (1:400, rat, Millipore), and CD68 (1:100, mouse, AbboMax).

At week 10 post-surgery, sciatic nerves in the sham, suture, fibrin, and DNNA groups were harvested and fixed. H&E and Masson's Trichrome staining were conducted as mentioned before. For the IF staining, nerves were dehydrated in 30% sucrose overnight, embedded into optimal cutting temperature (OCT, Fisher HealthCare) compound, and cut into 10 μm sections for the staining of αSMA (1:200, mouse, Sigma-Aldrich), CD68, TUBB3, and MBP. For semiquantitative analysis of collagen, αSMA, CD68, and TUBB3 positive areas, randomly selected Masson's Trichrome and IF images from each group were split into three channels (red, green, and blue) in ImageJ software. By adjusting the threshold, target areas were measured and divided by the whole image area to calculate the target area ratios.

Nerve segments distal to the injury site were isolated and fixed with 2% glutaraldehyde and 2% paraformaldehyde in 0.1 M cacodylate buffer (pH 7.2). They were then transverse sectioned into 1 μm sections for toluidine blue staining and 100 nm sections for TEM using a Leica UC7 microtome. Toluidine blue images were observed with a brightfield microscope (DMil, Leica). After a staining with 2% Uranyl Acetate and Reynolds Lead Citrate, sections for TEM were observed on a Tecnai G2 Spirit TWIN (Thermo Fisher Scientific) operating at 80 kV. Thirty images from 3 rats (ten images from each rat) from each group were randomly captured, and 300 nerve fibers were randomly selected for neural morphological analysis. The areas of axons and nerve fibers and the myelin thickness were measured using ImageJ. The area-based G ratio was further determined as the ratio of the axon area to the nerve fiber area.

### Statistical Analysis

All quantitative data were expressed as the mean±standard deviation (SD). Statistical analysis was also performed. In experiments with two groups, Student's t-tests were used, while in experiments with more than two groups, one-way analysis of variance ANOVA was used with Tukey post-hoc tests for statistical analysis. Differences with p<0.05 were denoted as *, while p<0.01 was denoted as **, p<0.001 was denoted as ***, p<0.0001 was denoted as ****, and ns indicated not significant.

### Results

FIG. 1 shows the concept and design of the catechol-based DNNA. HA is an important endoneurial ECM component, which can reduce scar formation in peripheral nerve injuries (Ikeda, et al. (2003) Nature Biotechnol., 21:526; Suri, et al. (2010) Tissue Engr. Part A, 16:1703), and was chosen as the polymer backbone for catechol-functionalization. It is modified by dopamine-isothiocyanate to obtain Cat and TU groups simultaneously (HA-TU-Cat in FIG. 1A). After oxidation, HA-TU-Cat crosslinks quickly through uniform and strong thiourea-quinone couplings, forming the first network (FIG. 1B). DPN, a nerve-specific biomaterial with abundant bioactive peptides and growth factors, is then incorporated. DPN self-assembles physically into the second network. The two networks covalently interact with each other through quinone in HA-TU-Cat and nucleophiles in DPN, forming the DNNA. When the DNNA is applied to the injured nerve ends, it connects nerve ends through quinone bonding to amines and thiols on the tissue surface covalently or noncovalently through hydrogen bonding (FIG. 1C). The two interacted polymer networks in the adhesive serve three important roles: 1) HA-TU-Cat ensures strong hydrogel adhesion and cohesion, feasible curing time for clinical application, and reduced quinone accumulation; 2) DPN provides abundant bioactive proteins, growth factors, and ECM components for axon regrowth; 3) interactions between HA-TU-Cat and DPN further reduce quinone amount and improve hydrogel cohesion, which helps resist failure and crack formation of the bulk matrix and increase adhesion strength, thereby.

Based on the design concept, the detailed fabrication and characterization of the dual-network nerve adhesive is described. Catechol-modified HA was prepared in two ways for comparison, namely, the Schiff base condensation and the isothiocyanate-amine coupling. The Schiff base was formed between the aldehyde-modified HA and dopamine, resulting the catechol-modified HA conjugates (HA-Cat) (Zhou, et al. (2020) ACS Appl. Mater. Interfaces 12:18225). Its successful synthesis was confirmed by ¹H Nuclear Magnetic Resonance (¹H NMR) , as indicated by the presence of catechol aromaticproton peaks at 8 ~7 ppm and catechol methylene-proton peaks at δ 3.1 and 2.8 ppm. The catechol group grafting ratio in HA-Cat was about 40%. In the second approach, the dopamine-isothiocyanate derivative was synthesized (Xu, et al. (2016) Biomaterials Sci., 4:1726), which was then grafted onto the HA-adipic dihydrazide (HA-ADH) backbone via isothiocyanate-amine coupling, thereby producing HA-TU-Cat (FIG. 1A). The ¹H NMR of HA-ADH, dopamine-isothiocyanate, and HA-TU-Cat were determined. The degree of substitution (DS) of catechol groups in HA-TU-Cat was determined to be about 20%.

Decellularized porcine nerves were prepared based on protocols (Xue, et al. (2022) ACS Appl. Mater. Interfaces, 14:8693). Hematoxylin and eosin (H&E) staining revealed loosened matrix structures of nerves and absent nuclei after decellularization. The deoxyribonucleic acid (DNA) content in porcine native peripheral nerves (NPN) decreased from 514±77 ng/mg to 39±12 ng/mg after decellularization, which met the international requirement of 50 ng/mg (FIG. 1D) (Crapo, et al. (2012) Biomaterials 33:3539). The collagen and glycosaminoglycan (GAG) concentrations slightly decreased, but no significant differences were detected (FIG. 1D). All these results proved successful decellularization and collagen/GAG maintenance of porcine peripheral nerves.

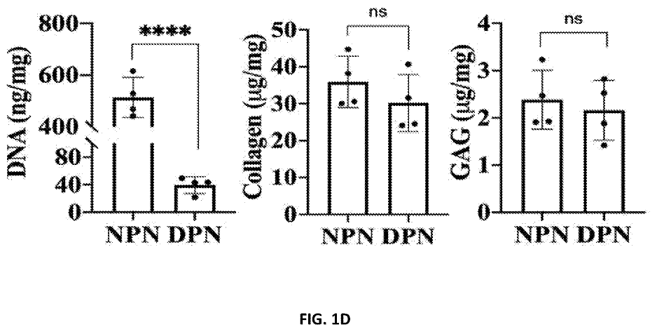

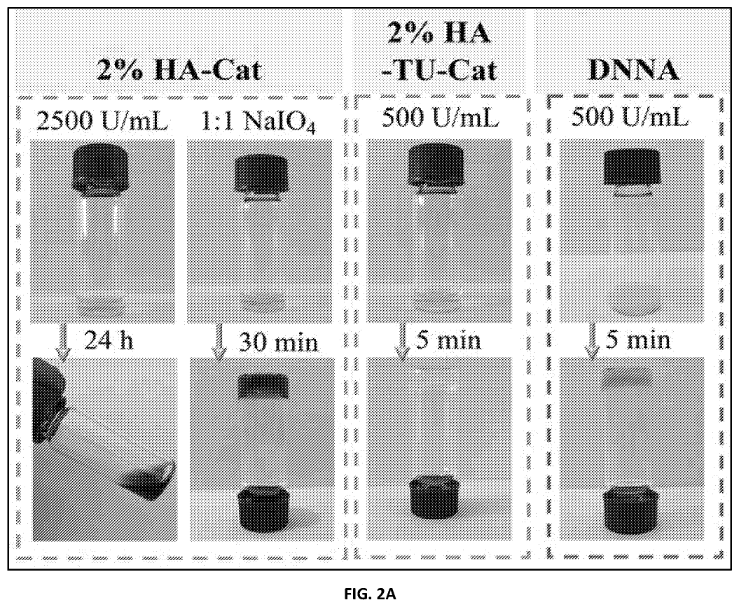

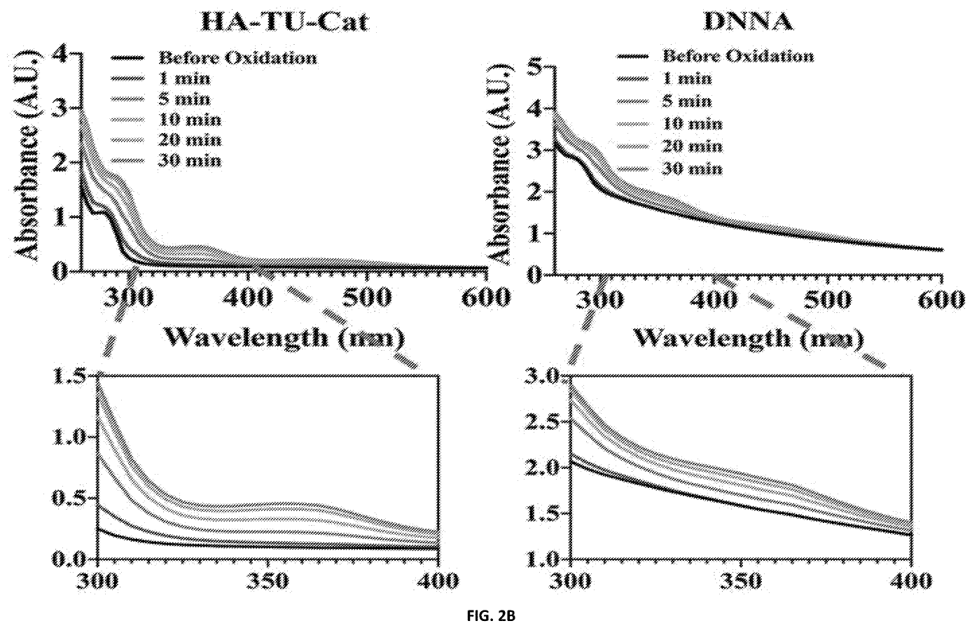

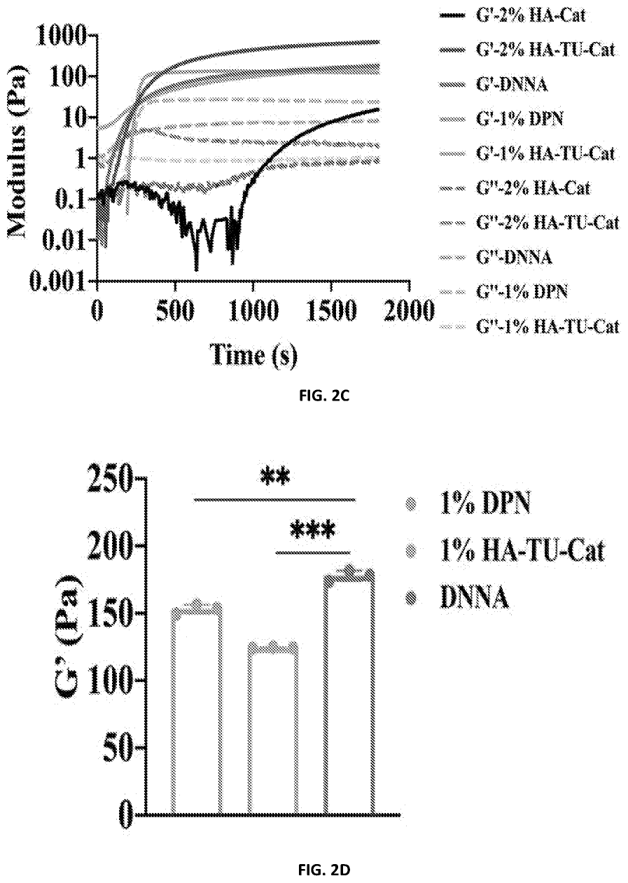

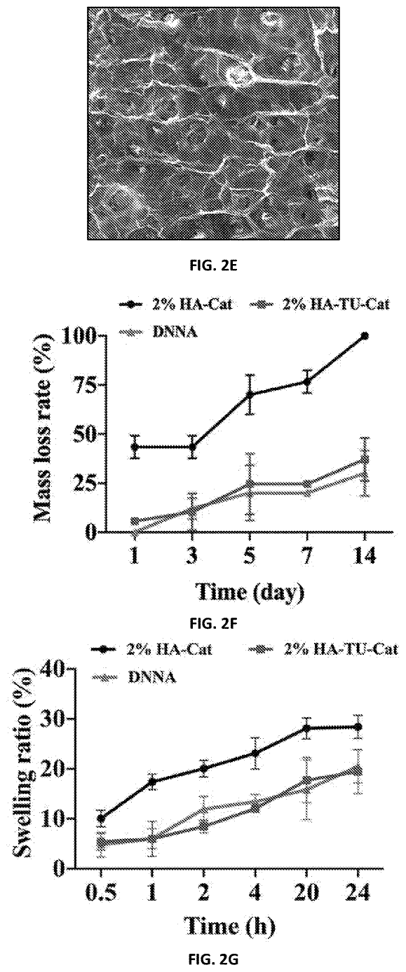

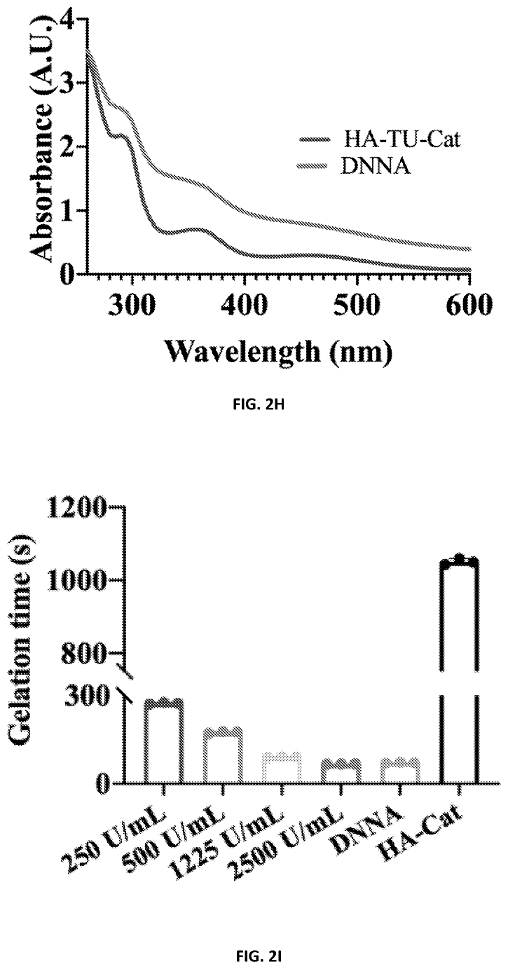

In order to produce in situ curable adhesive hydrogels, oxidation is required to induce crosslinking of catechol-based polymers and to connect tissues covalently. In this study, both a chemical oxidizing agent, i.e., sodium periodate (NaIO₄), and an enzymatic oxidizing agent, i.e., mushroom tyrosinase (MT), were implemented. Compared to NaIO₄, MT is advantageous due to its high specificity and biocompatibility (Jonker, et al. (2015) Adv. Mater., 27:1235). 2500 U/mL MT was added to induce HA-Cat (2%) gelation. After being incubated at 37 °C for 24 hours, the color of the HA-Cat solution became dark (FIG. 2A). However, hydrogel formation was not noticed. It was then switched to NaIO₄ to accelerate catechol oxidation, and HA-Cat gelated after 30 minutes of incubation at room temperature with a 1:1 molar ratio of NaIO₄ to catechol groups. In contrast, successful gelation of HA-TU-Cat (2%) and DNNA (1% HA-TU-Cat+1% DPN) was induced after a 500 U/mL MT addition and incubation at 37 °C for 5 minutes, indicating quick gelation behavior and feasibility for surgical application. Different from dark color of the HA-Cat hydrogel, the HA-TU-Cat and DNNA hydrogels were greyish, indicating less accumulated quinone groups (FIG. 2A). It indicated that quinone-thiourea couplings could reduce quinone amount in the matrix compared to quinone-quinone complex.

To further unveil the role of DPN on quinone consumption in DNNA, ultraviolet-visible (UV-vis) spectroscopy was recorded before and after the addition of MT. As shown in FIG. 2B, the quinone peak (~365 nm) appeared 10 minutes after the addition of MT and became stronger throughout the gelation time in HA-TU-Cat. Since HA-TU-Cat and DNNA gelated with the participation of quinone groups in 5 minutes (as shown by successful gelation within 5 minutes in FIG. 2A), the catechol group was speculated to turn into quinone after the addition of oxidants but was reduced back to catechol when bonding covalently with TU groups. TU could delay and protect catechol from overoxidation and cytotoxicity from quinone thereby. However, MT oxidized the catechol into quinone continuously, even after the depletion of TU. Thus, the quinone peak became stronger with increasing oxidation time. In regards to the DNNA with a decellularized matrix, an inconspicuous quinone peak appeared 30 minutes after the addition of MT (FIG. 2B) and stayed low even after a 3-hour reaction, compared to HA-TU-Cat (FIG. 2H). This result indicated that, compared to HA-TU-Cat, the covalent bonding of amines/thiols inside DPN with quinone could further decrease quinone amount, which was favorable for adhesion enhancement and limiting reactive oxygen species.

The modulus of adhesive hydrogel was investigated to determine its cohesion. According to the time sweep rheological analysis (FIGS. 2C), 2% HA-TU-Cat displayed quicker gelation behavior and higher storage modulus (G′, 45 times) than those of 2% HA-Cat hydrogels, even though the DS of HA-Cat was twice that of HA-TU-Cat. The differences in the gelation and mechanical properties of HA-Cat and HA-TU-Cat were attributed to different gelation mechanisms, where HA-Cat crosslinked through quinone self-polymerization, while HA-TU-Cat crosslinked through quinone-thiourea couplings, which were more uniform and efficient (Xu, et al. (2020) Sci. Transl. Med., 12:eaba8014; Schiessl, et al. (2005) Zeitschrift für anorganische und allgemeine Chemie, 631:2812). The addition of DPN into HA-TU-Cat resulted in a significantly higher storage modulus of DNNA (1% HA-TU-Cat+1% DPN) than that of 1% DPN and 1% HA-TU-Cat individually (FIG. 2D). This indicated that the DNNA was more than just a physical mixture of DPN and HA-TU-Cat. Networks formed by DPN and interactions between DPN and HA-TU-Cat networks strengthened the cohesion of DNNA hydrogels. The MT concentration did not show significant influence on the G′ of HA-TU-Cat, but a higher MT concentration did shorten the gelation time (the time when storage modulus was higher than loss modulus) from around 5 minutes to 1 minute when MT concentration increased from 250 U/mL to 2500 U/mL (FIG. 2I). 500 U/mL MT was chosen to allow enough operation time as well as potential adjustment during surgery.

The gelation time can be further regulated through MT concentration according to the surgical needs. But for commercial fibrin glues, they crosslink rapidly (within 1 minute), which restricts any further position adjustments once applied. For HA-Cat with different NaIO₄ concentrations, the 1:0.5 molar ratio of catechol to NaIO₄ illustrated similar storage modulus and loss modulus (G″), while the 1:1 ratio demonstrated higher G′ than G″. Thus, the 1:1 ratio was chosen in further studies.

The morphologies of HA-Cat, HA-TU-Cat, and DNNA were evaluated by scanning electron microscopy (SEM), shown in FIG. 2E. All hydrogels showed porous and interconnected structures. The incorporation of DPN seemed to introduce more fiber-like structures into DNNA. The HA-Cat fully dissociated within 14 days in vitro, which was faster than HA-TU-Cat and DNNA (FIG. 2F). HA-TU-Cat and DNNA illustrated comparable stability, with around 25% mass loss after 14 days. Hydrogel hydrolytic stability normally depends on the cleavage of chemical bonds among polymer backbones. The quinone-quinone oligomerization structure in HA-Cat is random and inefficient. Therefore, it renders the HA-Cat more hydrolytically labile, which leads to a faster dissociation and mass loss of HA-Cat. However, quinone-thiourea couplings in HA-TU-Cat and DNNA are more stable. All hydrogels exhibited gradual swelling over time and reached equilibrium after 20 hours (FIG. 2G). HA-Cat had a higher swelling ratio than that of HA-TU-Cat and DNNA. The stability and swelling results were consistent with the random oligomerization structure in HA-Cat and the uniform quinone-thiourea couplings in HA-TU-Cat and DNNA.

Nerves are subjected to different deformations during daily walking and exercise, including shear and stretch. The adhesive performances of DNNA were evaluated through lap shear and tensile tests to determine its shearing and tensile resistance, respectively, and compared to commercial fibrin glues.

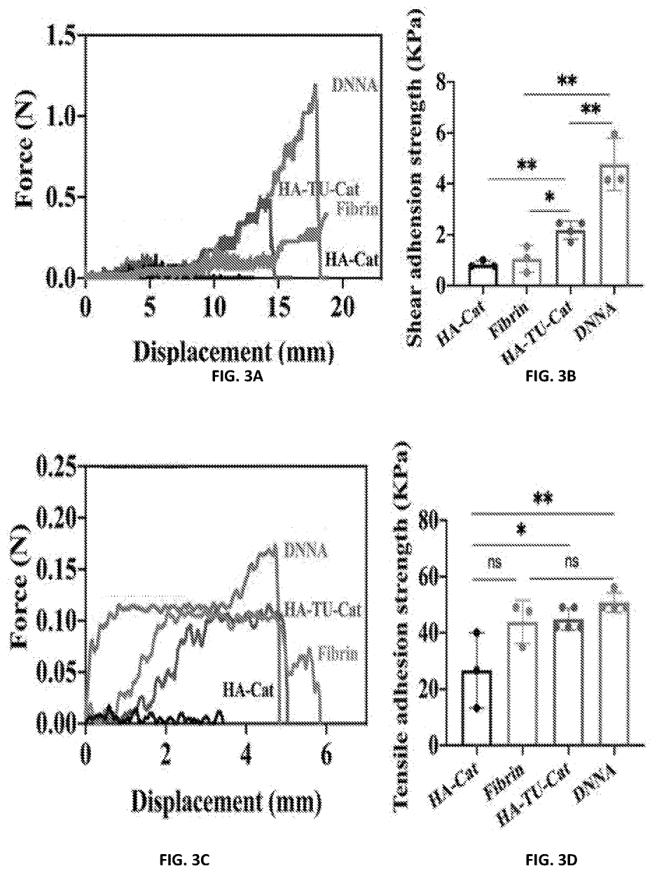

A lap shear test based on fresh porcine skins was applied to assess the ability of adhesives to withstand the stress in a plane when shear forces displaced two skins joined by the adhesive. FIG. 3A shows the displacement-force curves of HA-Cat, HA-TU-Cat, DNNA, and commercial fibrin glues. DNNA demonstrated a remarkably increased adhesion force, as well as failure displacement, compared to its counterparts. The adhesion strength was further calculated. DNNA illustrated 5 times higher values compared to fibrin glue and HA-Cat (FIG. 3B). In comparison to HA-TU-Cat, the addition of DPN significantly improved the shear resistance ability of the DNNA (FIG. 3B). The successful function of adhesives not only depends on the adhesion between adhesives and tissues but also the cohesion among the adhesive matrix (Nam, et al. (2021) Chemical Rev., 121(18): 11336-11384). On one hand, DPN introduced more nucleophiles into the DNNA, which increased its hydrogen bonding with tissues. On the other hand, the addition of DPN strengthened the cohesion of DNNA hydrogels. In DNNA, besides quinone-thiourea couplings, DPN self-assembled and neutrophils from DPN could covalently bond with oxidized catechol from HA-TU-Cat. These reactions greatly enhanced DNNA cohesion and adhesion, compared to HA-TU-Cat.

Stretch is another common deformation of nerves, especially for transected nerves undergoing strong retraction due to the elastic endoneurium (Lee, et al. (2020) Sci. Rep., 10:1). A tensile test was used to pull two nerves connected by adhesives to determine stretching adhesion forces. The DNNA showed a higher failure force and adhesion strength than HA-Cat, but no significant difference was observed between HA-TU-Cat and DNNA (FIG. 3C and FIG. 3D). Although there was weak shear adhesion performance, the tensile force and tensile adhesion strength of fibrin were high and had no statistical differences with those of DNNA (FIG. 3C and FIG. 3D). The enhanced adhesion performance of DNNA was not shown in the tensile tests due to smaller contact area and less hydrogel amount applied (200 μL for the lap shear test and 20 μL for the tensile test), compared to the lap shear test through porcine skins.

To qualitatively show the adhesion performance of the DNNA for peripheral nerve anastomosis, a rabbit sciatic nerve was transected into three sections, and the DNNA was applied at the nerve ends to adhere them. After gelation at 37 °C for 5 minutes, the reconnected nerve was hung by a pair of tweezers, and the DNNA could successfully bear the reconnected nerve weight. In situ adhesion of a transected rabbit sciatic nerve was also demonstrated after 5 minutes at room temperature, and the adhesive nerve can even withstand a slight stretch by a blade. DNNA with green food dye was applied onto the surface of porcine skins. The gelated DNNA showed strong adhesion to the porcine skin, as evidenced by the capability to withstand external deformations like twisting, soaking in PBS for 30 minutes, and flushing under running water for 5 minutes. Longer time in PBS was shown. In addition to porcine skins, adherent hydrogels on slides were also able to bear flushing under water.

Overall, these quantitative results demonstrated stronger adhesion of DNNA compared to fibrin glues. The addition of DPN further significantly improved hydrogel shearing adhesion resistance. The robust adhesion performance of the DNNA in tissues, slides, and wet environments was also confirmed qualitatively.

SCs and peripheral axons are two of the most important cell types during peripheral nerve injury and repair processes. SCs are responsible for myelin sheath and axonal debris clearance, providing essential trophic support for injured neurons and further remyelination. Peripheral neuron axonal outgrowth and myelination reflect the nerve injury repair status. The bioactivity and biocompatibility of the decellularized matrix in DNNA were evaluated through SC proliferation and neurite outgrowth in vitro.

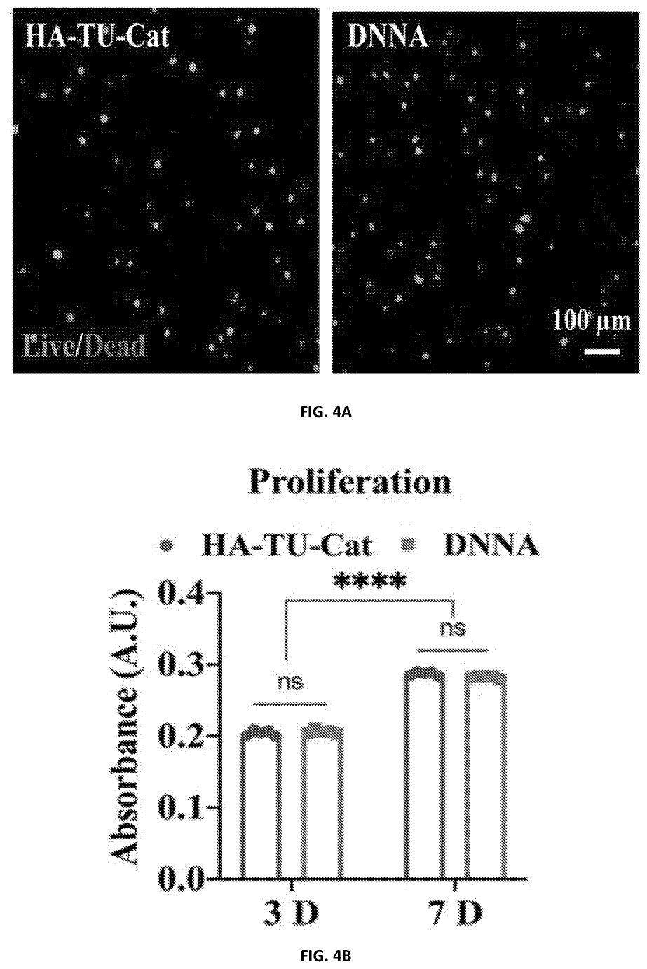

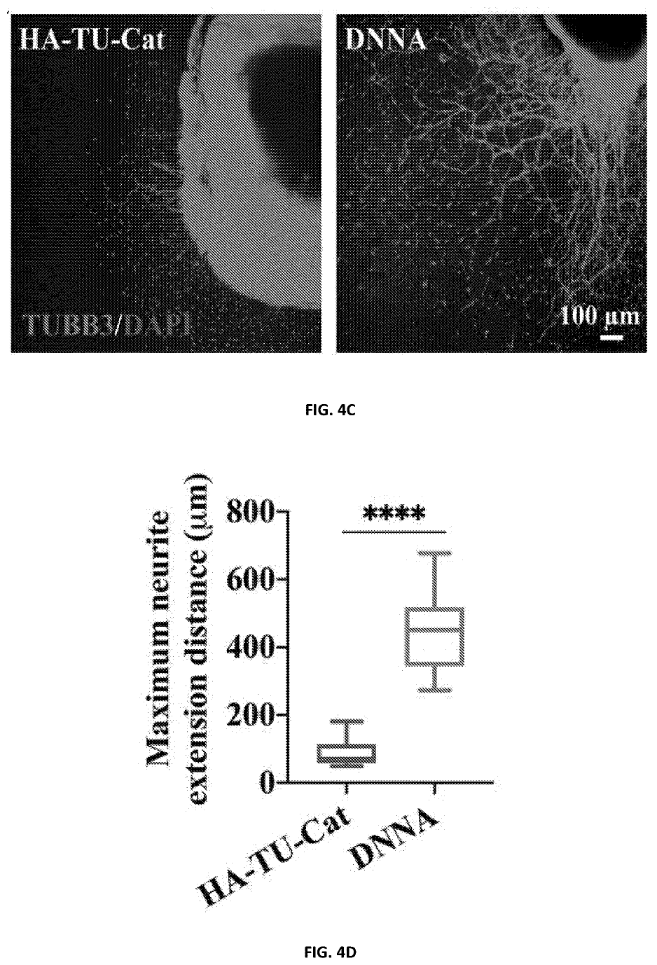

Primary rat SCs were encapsulated within HA-TU-Cat and DNNA hydrogels, and they were alive in both hydrogels after 7 days (FIG. 4A). A spherical cell shape without spreading was also noticed in both hydrogels. SCs proliferated significantly from day 3 to day 7 in both HA-TU-Cat and DNNA, and the addition of DPN did not significantly affect cell growth rate (FIG. 4B). The main reason DPN incorporation did not efficiently promote SCs spreading and growth was that hydrogel mechanics regulated cell behaviors besides hydrogel composition. SCs were encapsulated within two concentrations of commercial rat collagen hydrogels (0.15% and 0.4%) and decellularized rat nerve hydrogels (0.4% and 0.9%) (Xue, et al. (2022) ACS Appl. Mater. Interfaces, 14:8693). It was found that SCs in two concentrations of collagen hydrogels had the spherical shape without spreading. For decellularized nerve hydrogels, 0.9% ones with storage moduli around 100 Pa also illustrated spherical cell shape and lower proliferation rate, while cells in 0.4% ones with moduli around 20 Pa illustrated strong SC remodeling capacities, with spreading morphology and higher cell proliferation rates. The results indicated that, apart from the material composition, hydrogel mechanics also played important roles in regulating SC behaviors. In the current study, both DNNA and HA-TU-Cat had storage moduli above 100 Pa, which may lead to weak cell remodeling abilities. Besides, it was reported that despite round morphology of encapsulated SCs in the decellularized matrix in vitro, SCs illustrated high viability and maintained functionality after transplantation in vivo (Cerqueira, et al. (2018) Biomaterials 177:176).

In contrast to SC growth behaviors, the addition of DPN remarkably improved axonal outgrowth from the DRGs (FIG. 4C). Tubulin beta III (TUBB3) stained axons had longer extension distances and more branches in the DNNA than those in HA-TU-Cat hydrogels. A semi-quantitative measurement illustrated 5 times longer neurite length in the DNNA than those in HA-TU-Cat hydrogels without the decellularized matrix (FIG. 4D). Consistent with previous studies, DPN facilitated DRG neurite outgrowth and hydrogel composition was dominant in regulating neurite behaviors, compared to hydrogel mechanics (Xue, et al. (2022) ACS Appl. Mater. Interfaces, 14:8693). Bioactive ECM components in the DPN and peptides/growth factors generated after hydrogel degradation were reported to play important roles. For example, it was suggested that collagen IV in DPN could promote neurite attachment and growth through integrin ITGA1 (Sun, et al. (2020) Biomaterials 258:120289). Laminin was also abundantly found in DPN and played positive roles in neurite extension (Xue, et al. (2022) ACS Appl. Mater. Interfaces, 14:8693).

The results here verified good biocompatibility of the adhesive and positive role of bioactive components from decellularized nerve matrix, which could facilitate axonal outgrowth and branching.

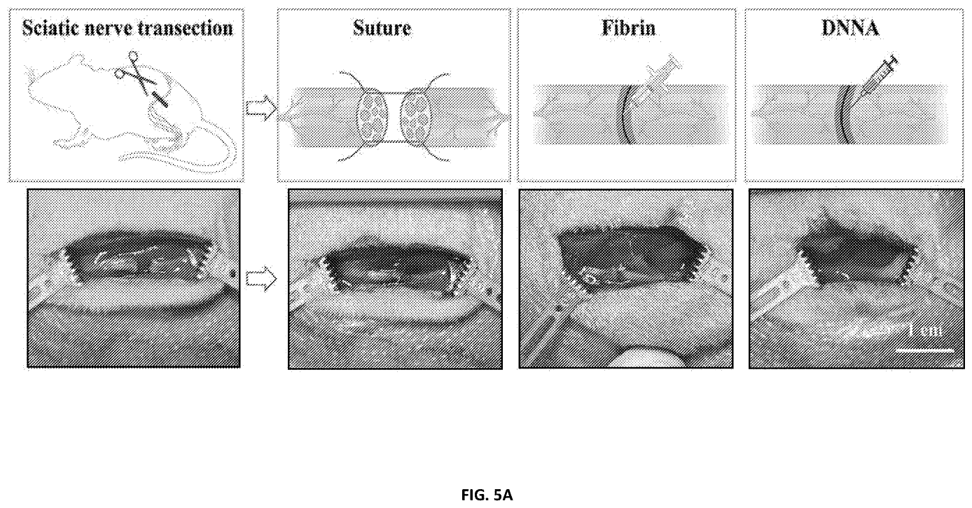

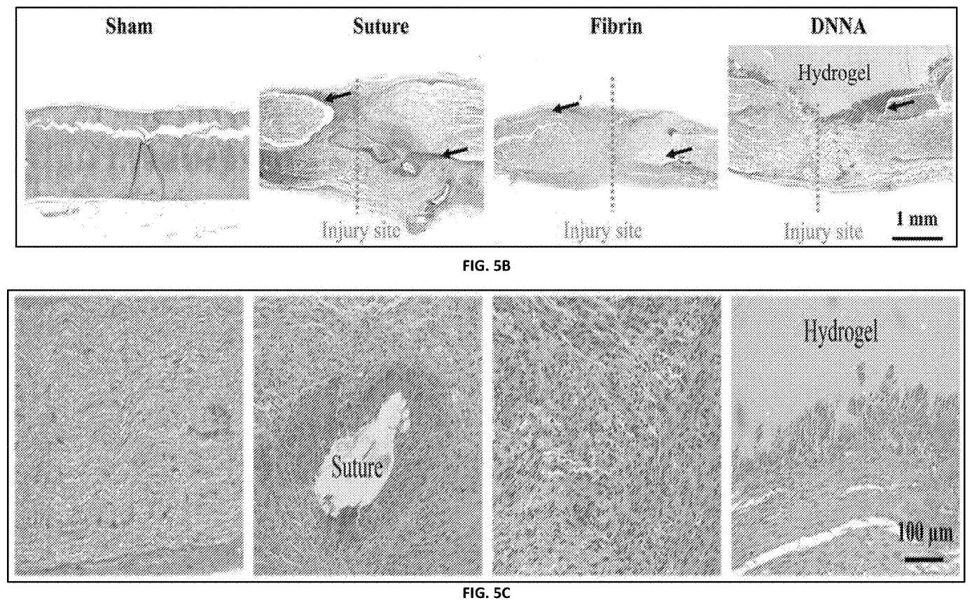

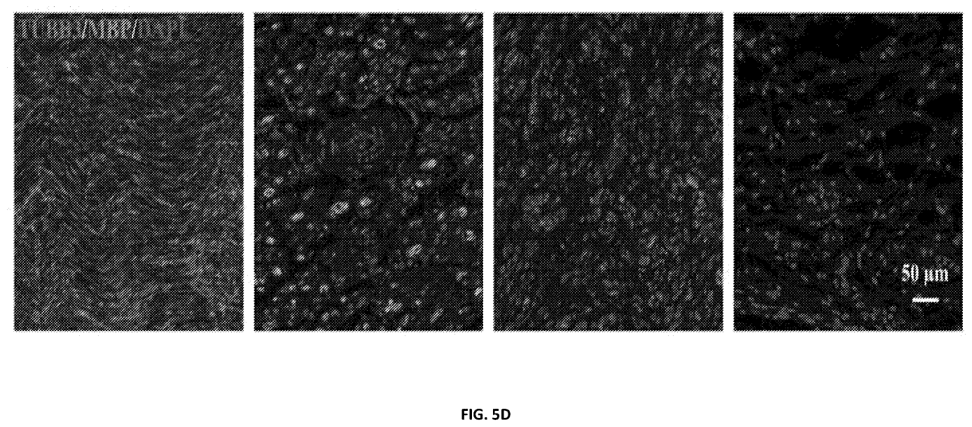

To test the therapeutic efficacy of DNNA and compare it with a clinical standard suture and a commercial fibrin glue, the adhesives were applied for nerve anastomosis in a rat-based nerve transection model (FIG. 5A). After 10 days of surgery, the injured nerves were collected for short-term histological analysis. Masson's Trichrome (FIG. 5B) and H&E (FIG. 5C) staining of harvested sciatic nerves was performed. In FIG. 5B, collagen-rich fibrotic tissues were found in the suture and fibrin repaired nerves, while there were much less in the DNNA group (indicated by the arrows in FIG. 5B). Besides, compared to fibrin and DNNA adhesives, the suture application caused nerve structure interruption and axon mis-direction. Residual sutures and hydrogels without complete degradation were observed in suture and DNNA treated nerves, respectively. According to the H&E staining, foreign body granulomas with abundant nuclei surrounding the residual suture were noted in the suture group (FIG. 5C). There were also some foreign body reactions at the boundary between the hydrogel and the nerve in the DNNA group (FIG. 5C). For fibrin treated nerve, although no obvious hydrogel residue was detected, many proliferative fibroblasts disrupted nerve fascicle structures (FIG. 5C). Moreover, it was worth noting that TUBB3 and myelin basic protein (MBP) staining showed little axon and myelin debris in our DNNA adhesive at the injury site. In contrast, plentiful remaining myelin and axon debris still existed in the suture and fibrin treated nerves (FIG. 5D).

During the early stage of nerve injury and repair, macrophages help clear axon and myelin debris to create a microenvironment conducive for axonal regrowth and reconnection, which is an essential part of Wallerian degeneration (George, et al. (1994) J. Neurocytol., 23:657). But large numbers of macrophages can also lead to intraneural inflammation and fibrosis formation. CD68 staining was applied to examine any difference of macrophages in different treatments. No significant difference was shown in three groups regarding macrophage intensity and number. One possible explanation for different debris clearance results is that similar amounts of macrophages infiltrated but took different responsibilities in different groups. The infiltrated macrophages contributed to fibrosis induction in the suture and fibrin repaired nerves, whereas with the regulation of bioactive decellularized matrix, the macrophages helped with the Wallerian degeneration in the DNNA group. Those macrophage contribution differences may be caused by the treatment methods and hydrogel materials applied. Suture treatment had secondary damage and foreign body reactions, and the fibrin persistence at injury sites was reported to be linked to increased fibrosis (Barnes, et al. (2011) Biomaterials 32:137). It was demonstrated that the rate of collagen synthesis increased in a three-dimensional fibrin matrix, as opposed to a collagen matrix (Coustry, et al. (1990) FEBS Lett., 262:339). In contrast, the incorporated decellularized matrix in the DNNA has been reported to positively regulate the immune responses after nerve injury (Wang, et al. (2022) Biomaterials 280:121251; Sun, et al. (2020) Biomaterials 258:120289). It was probable that the body took embedded signaling cues from the DPN and used these cues to direct the in vivo remodeling process.

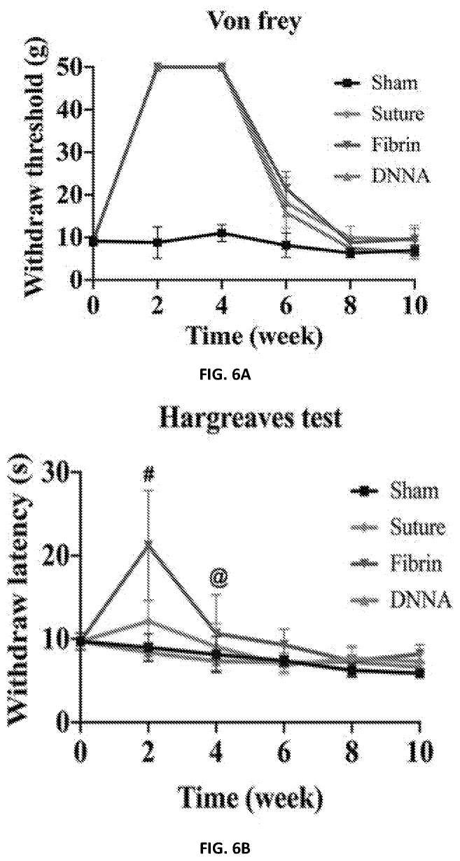

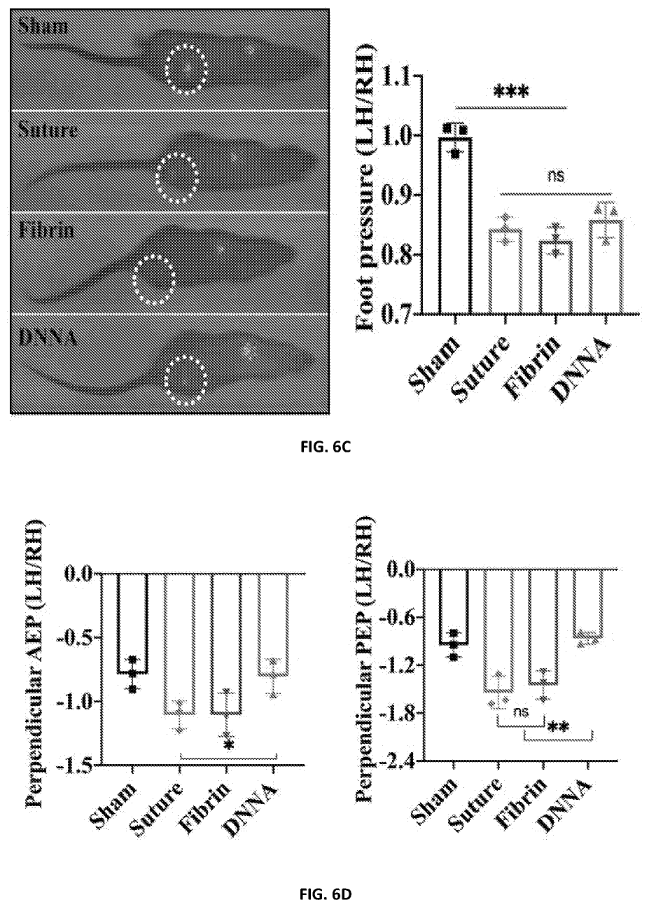

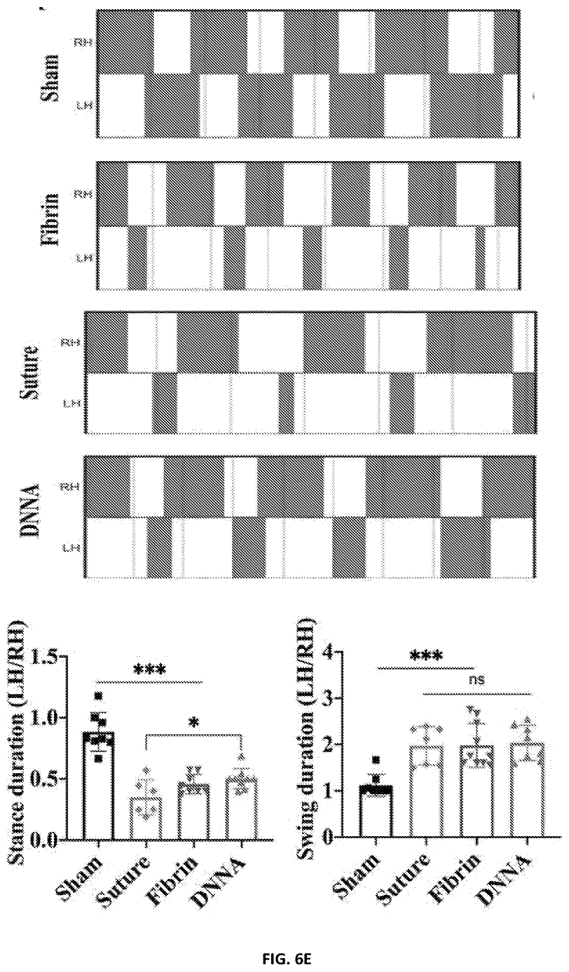

Sensory and motor function recoveries are important aspects of nerve regeneration after transection. Here, mechanical and thermal sensory functions of rats were tested by von Frey and Hargreaves tests, respectively. All animals started to recover partial mechanical sensation after 6 weeks from surgery (FIG. 6A). The foot withdrawal thresholds in the rats treated by DNNA were lower, but no significant difference was found in three different strategies. The mechanical sensory function gradually recovered to the level of the sham control in all injured rats over time. The thermal sensation of rats treated by DNNA was found to recover to the level of the sham control 2 weeks post-surgery and kept a withdrawal latency time comparable to sham rats after that (FIG. 6B). A statistical difference was observed between DNNA and fibrin glue groups at weeks 2 and 4, indicating that DNNA improved early thermal function recovery. Besides, faster thermal sensation recovery than mechanical recovery was noticed in all groups. The mechanisms behind are still not clearly understood (Gorodetskaya, et al. (2009) J. Neurophys., 102:3129; Jankowski, et al. (2009) J. Neurosci., 29:1636).

A rat-walking apparatus system was used to evaluate the recovery of locomotor functions by comparing the experimental foot (left hind in the system, LH) with the contralateral healthy foot (right hind in the system, RH) at week 10 (Croen, et al. (2021) J. Orthoped. Res., 39:2243; Mendes, et al. (2015) BMC Biol., 13:1). The footprint intensity and foot pressure of transected nerve rats were significantly lower than those of sham group, and no statistical difference was noticed among nerve anastomosis groups for the foot pressure (FIG. 6C). Rat body wobble was reflected by stance trace. Perpendicular anterior extreme position (AEP) and posterior extreme position (PEP) recorded the foot position relative to the body center of paw touchdown and stance end, respectively (Mendes, et al. (2013) elife 2: e00231). Rats treated by DNNA illustrated comparable perpendicular AEP and PEP values with those of the sham, while they showed obvious body wobble, with AEP and PEP far from the body axis in the suture and fibrin glue groups (FIG. 6D). Unequally distributed gait patterns with shorter stance phases (white squares) and extended swing durations (gray squares) were observed in all transected nerve animals (FIG. 6E). This indicated hesitations of paw touchdown after nerve transection injury. However, a significantly longer stance duration was shown in DNNA repaired rats, indicating better lower limb motor function recovery. Differences in the swing duration among the groups were statistically insignificant (FIG. 6E).

Herein, myelin and axon debris clearance has been demonstrated in DNNA treated rats 10 days after surgery, which may be beneficial for long-term nerve regeneration. To further determine and verify the long-stage efficacy of DNNA treatments, nerves in different groups were harvested 10 weeks postoperatively. Here, the reconnection and remyelination of nerves were evaluated systematically.

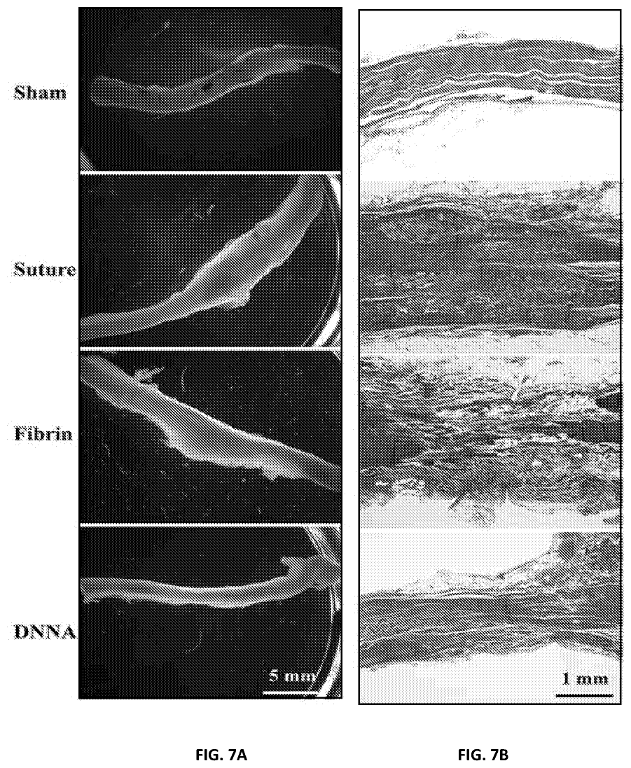

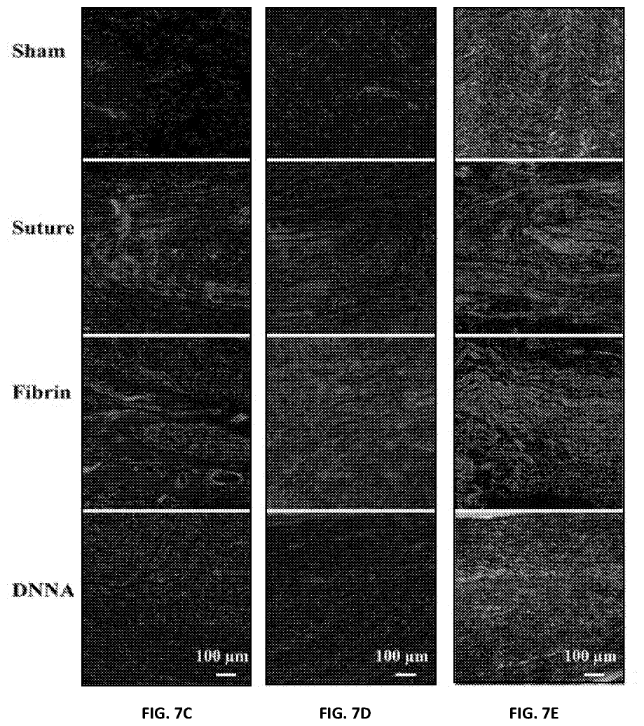

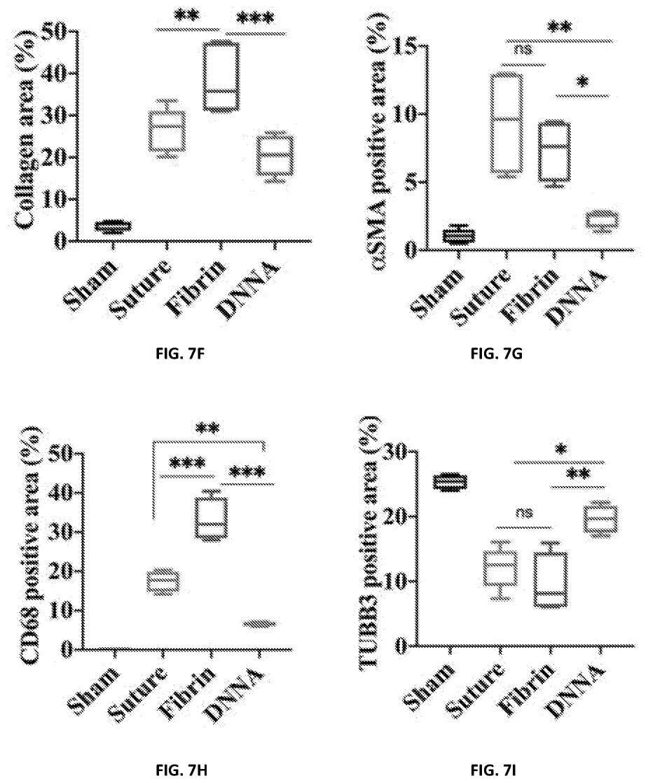

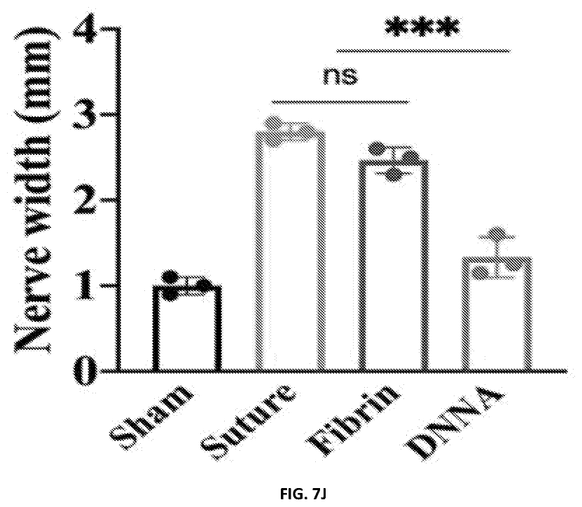

According to nerve photographs, nerves were uniform in diameter with complete hydrogel degradation and without evidence of injury in the DNNA group, while suture and fibrin treated nerves showed bulges at the injury sites (FIG. 7A). To further evaluate the structure continuity and connective tissue deposition of the treated nerves, Masson's Trichrome and H&E staining were performed (FIG. 7B). Dense scar tissue surrounding the nerve trunk was distinguishable as bands of blue-stained collagen in the suture and fibrin groups, while there was much less collagen observed in the DNNA group (FIGS. 7B and 7C). Scar deposition also led to nerve thickening in the suture and fibrin repaired groups, which were twice larger in diameter compared to DNNA (FIG. 7J). In addition, the fibrosis in the suture and fibrin groups was more severe compared with that at 10 days postoperatively, indicating persistent immune reactions and collagen depositions. As for the nerve continuity, fibrosis and foreign body reactions led to granuloma formation, structure disturbance, and nerve fiber misdirection in the suture coapted nerves (FIG. 7B). Fibrin treated nerves showed intermittent nerve fiber distribution and poor reconnections with numerous connective tissues at the injury site. In contrast, directed nerve fiber regrowth and reconnection were demonstrated in the DNNA group, which was consistent with better sensory and motor function recovery described previously.

Myofibroblasts and macrophages are two important cells responsible for the excessive synthesis, deposition, and remodeling of collagens during fibrosis (Wynn, et al. (2016) Immunity 44:450). Immunofluorescence (IF) staining of alpha-smooth muscle actin (αSMA) showed a considerable increase of myofibroblast expression in the suture and fibrin groups, compared to the DNNA group (FIGS. 7C and 7G). CD68 IF staining was then performed to identify macrophages in sciatic nerves (FIG. 7D). Nerves from sham rats showed rare CD68 positive cells, while suture and fibrin groups exhibited markedly increased macrophage numbers, especially for the fibrin group. DNNA alleviated long-term inflammation, compared to suture and fibrin, indicated by the decreased CD68 intensity and positive area (FIG. 7H).

IF staining with TUBB3 and MBP was then performed to specifically assess axonal reconnection and myelination after nerve transection and repair (FIG. 7E). The connection of TUBB3 positive axons was diverted and blocked by the granuloma in suture group, and misdirection and misalignment of nerve fibers were also noticed. Discontinuous and mussy axons were shown in fibrin group. In contrast, nerves treated by DNNA illustrated directed regrowth, and the density of the nerve fibers was significantly higher than that in the suture and fibrin groups (FIG. 7I). Most importantly, more MBP positive myelin was observed in the DNNA group at the injury site, which was also confirmed by higher magnification images.

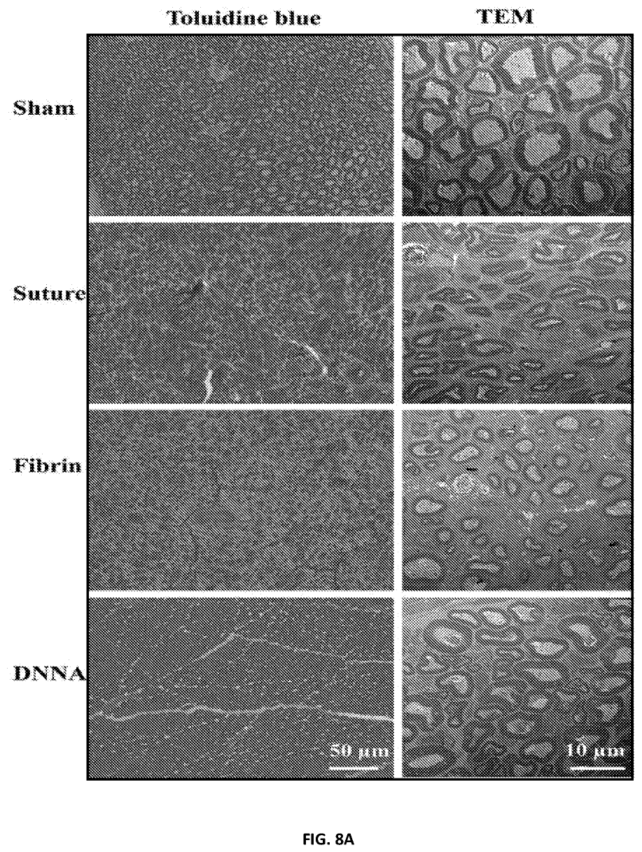

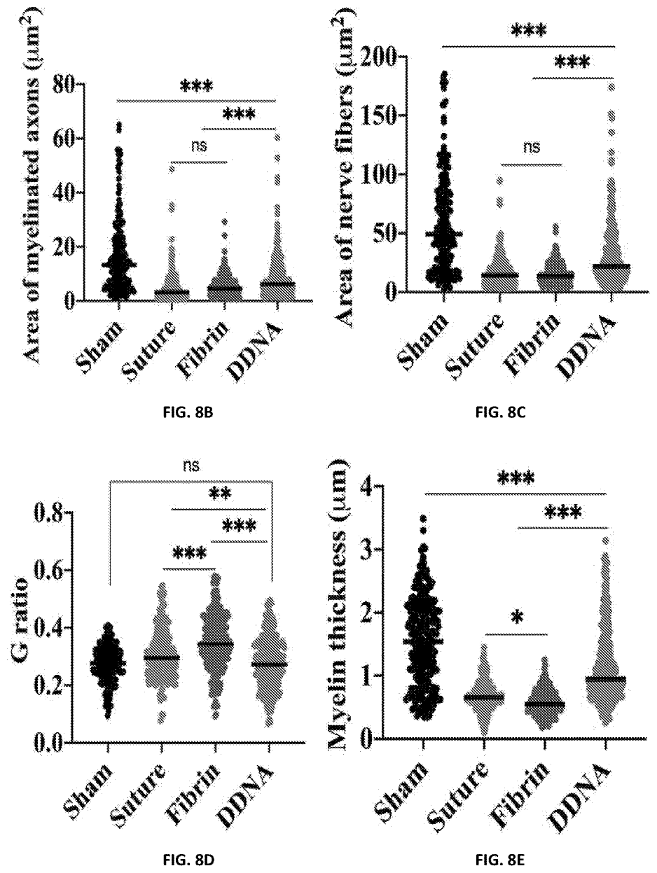

The remyelination of regenerated nerves was further studied through toluidine blue staining and transmission electron microscopy (TEM) (FIG. 8A). Nerve samples were collected from the distal ends and imaged in the cross-sectional directions. Toluidine blue images revealed much denser and larger neural fibers in the DNNA group than those in the suture and fibrin groups, indicating better regeneration and remyelination of DNNA treated nerves. However, all injured nerves had smaller nerve fibers and decreased myelin sheath thicknesses than the sham control. TEM examination also showed the presence of clusters of large, myelinated fibers in the DNNA group, in contrast to thin and sparse myelinated fibers in the suture and fibrin groups. Statistical analysis based on TEM images confirmed significantly increased sizes of myelinated axons (axon area in myelinated nerve fibers) and nerve fibers (including axon and myelin sheath) after treatment by DNNA, compared to suture and fibrin glues, though still smaller than native ones (FIGS. 8B and 8C). The G ratio of regenerated myelinated fibers, defined as the ratio of axonal area to nerve fiber area used for indicating optimal function and structure of neural myelination, was also calculated (Chew, et al. (2007) Adv. Funct. Mater., 17:1288). As shown in FIG. 8D, the G ratio distribution trend of DNNA repaired nerves was comparable to that of the sham group, and the value of the G ratio gradually decreased following the order: fibrin glue group (0.35±0.09)>suture group (0.30±0.09)>DNNA group (0.28±0.09)>sham nerve (0.27±0.07). In addition, the thickness of myelin sheaths from the DNNA group (1.15±0.60 μm) was about 2 times higher than those from the suture (0.67±0.23 μm) and fibrin glue (0.57±0.19 μm) groups (FIG. 8E).

In addition to fibrosis, macrophages also contribute to other pathogenic processes at the late stage of peripheral nerve repair. Persistent inflammatory responses with continuing macrophage presence impaired SC remyelination and suppressed nerve regeneration after injury (Buttner, et al. (2018) Aging Cell 17:e12833). Similar to results 10 days after surgery, the DNNA showed immune-regulatory behaviors in long term. It weakened intraneural inflammation and fibrosis, and enhanced nerve reconnection and remyelination, compared to suture and fibrin groups.

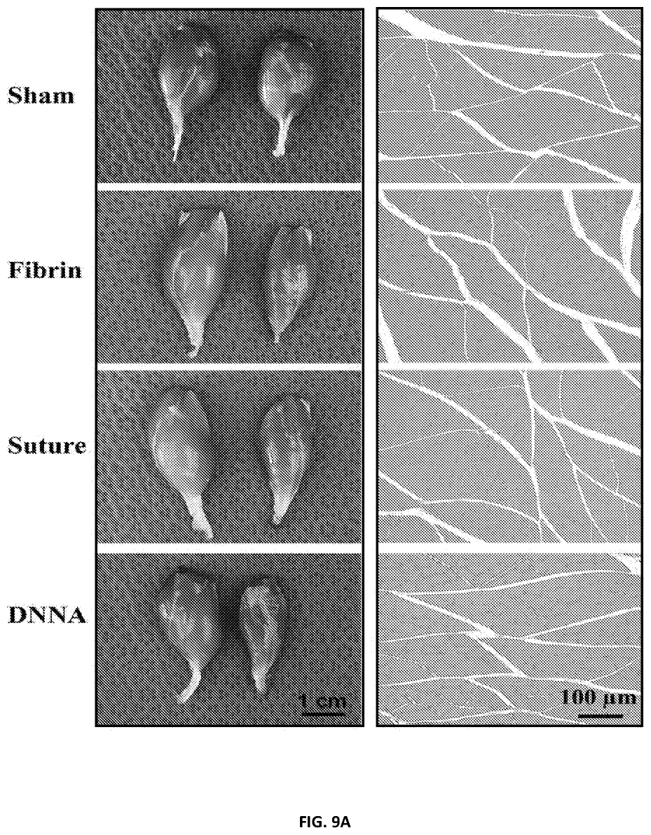

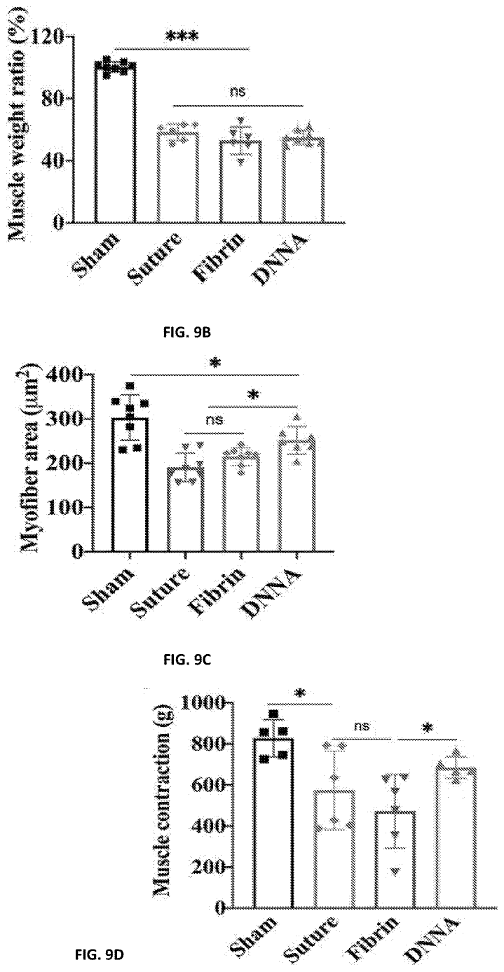

Nerve related muscle structure and function maintenance is also a crucial aspect for nerve regeneration. Thus, the weight, structure, and contraction force of the gastrocnemius muscles after treatment was tested. Gastrocnemius muscles were harvested 10 weeks post-operation (FIG. 9A), and the wet weight ratio (injured side/healthy side) was calculated. After injury and repair, the muscle weight was significantly decreased compared to sham group, but no statistical significance was noticed among different treatments (FIG. 9B). Next, the morphology of the gastrocnemius muscles was studied by the H&E staining (FIG. 9A). Of note, the DNNA group had significantly larger cross-sectional area of myofibers, compared to the suture and fibrin groups (FIG. 9C). The maximum muscle contraction force was an important indication of muscle reinnervation and function after injury. The DNNA group illustrated muscle contraction force close to the sham group, with low standard deviation, and was significantly higher than the fibrin group (FIG. 9D).

Taken together, the current design strategy of incorporating dopamine-isothiocyanate modified HA and decellularized matrix provides an efficacious adhesive hydrogel. The nerve adhesive was demonstrated to be superior, or at least comparable (in some aspects), to commercial fibrin glue and microsurgical suture for peripheral nerve transection treatment. It can also inspire the development of other tissue adhesive systems, like gastrointestinal defect sealing. In addition, DNNA may be used as a bioactive cargo delivery system (Warren, et al. (2021) APL Bioengr., 5:020901; Yao, et al. (2022) Adv. Sci., 2202102; Zhang, et al. (2020) Adv. Sci., 7:2002601; Yuan, et al. (2021) Biomaterials 279:121190). The DNNA could effectively connect the injured nerve ends and deliver cells and therapeutic agents at the same time to further enhance nerve regeneration. Adipose-derived stem cells (ADSCs) and SCs have been reported to enhance axonal growth and lead to improved nerve repair in vivo (Yuan, et al. (2021) Biomaterials 279:121190; Jahromi, et al. (2020) Mater. Sci. Engr., 109:110564; Sun, et al. (2019) Biomaterials 203:86). Drug-assisted treatments with neurotrophic factors, including nerve growth factors (NGF) and glial derived neurotrophic factors (GDNF), have potentials in the treatment of peripheral nerve injuries (Lackington, et al. (2019) J. Controlled Rel., 304:51; Fadia, et al. (2020) Sci. Transl. Med., 12: eaav 7753). They play key roles in proliferation of Schwann cells and the survival of sensory and motor neurons. Sustained delivery of other therapeutic agents like magnesium (Mg) ions or extracellular vesicles can also stimulate the nerve regeneration process and facilitate nerve recovery (Yao, et al. (2022) Adv. Sci., 2202102; Zhang, et al. (2020) Adv. Sci., 7:2002601; Yu, et al. (2021) Acta Biomaterialia 134:190).

To summarize, a bioactive, dual-crosslinked, and high-efficacy nerve adhesive for nerve transection treatment is described. The nerve adhesive exhibited fast and adjustable gelation behaviors and robust adhesion strength feasible for clinical applications. When applied to rat transected sciatic nerves, the adhesive significantly decreased intraneural inflammation and fibrosis, enhanced aligned axon connection and remyelination, as well as muscle function, compared to clinical suture and commercial fibrin treatments. These results collectively indicate that the nerve adhesive is an alternative to suture for nerve transection treatment.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A compound comprising a polymer linked to a phenol via a thiourea.

2. The compound of claim 1, wherein said polymer is a glycosaminoglycan.

3. The compound of claim 2, wherein said glycosaminoglycan is hyaluronic acid.

4. The compound of claim 1, wherein said phenol is a hydroxyphenol.

5. The compound of claim 4, wherein said hydroxyphenol is catechol.

6. The compound of claim 1, wherein said polymer is conjugated to the thiourea via a linker.

7. The compound of claim 1, wherein said phenol is conjugated to the thiourea via a linker.

8. The compound of claim 1, wherein the polymer is hyaluronic acid, wherein the phenol is catechol, wherein the hyaluronic acid is conjugated to the thiourea via a linker, and wherein said thiourea is conjugated to the catechol via a linker.

9. A composition comprising the compound of claim 1 and a carrier.

10. An adhesive comprising the compound of claim 1 crosslinked with decellularized peripheral nerve matrix.

11. An adhesive comprising a hyaluronic acid conjugate crosslinked with decellularized peripheral nerve matrix, wherein said hyaluronic acid conjugate comprises hyaluronic acid conjugated to a catechol via a linker comprising thiourea.

12. The adhesive of claim 11, further comprising a therapeutic agent.

13. The adhesive of claim 11, further comprising cells.

14. A method for producing an adhesive of claim 10, said method comprising mixing a compound of claim 1 and decellularized peripheral nerve matrix with an oxidizing agent.

15. A method for producing the adhesive of claim 11, said method comprising mixing a hyaluronic acid conjugate and decellularized peripheral nerve matrix with an oxidizing agent.

16. The method of claim 14, wherein said oxidizing agent is sodium periodate.

17. The method of claim 14, wherein said oxidizing agent is tyrosinase.

18. A method for adhering tissue within a subject, said method comprising administering the adhesive of claim 11 to the site for adhesion.

19. A method for treating a peripheral nerve injury, said method comprising administering the adhesive of claim 11 to the site of injury.

20. The method of claim 19, wherein said injury is a transection.
